SEQUENCE LISTING (28 sequences)

SEQ ID NO: 1 - protein, 8 aa, Artificial Sequence
  Description of Artificial Sequence Synthetic peptide
GPLGIAGQ

SEQ ID NO: 2 - protein, 6 aa, Artificial Sequence
  Description of Artificial Sequence Synthetic peptide
PLGLWA

SEQ ID NO: 3 - protein, 6 aa, Artificial Sequence
  Description of Artificial Sequence Synthetic peptide
PLGLGA

SEQ ID NO: 4 - protein, 6 aa, Artificial Sequence
  Description of Artificial Sequence Synthetic peptide
PLGLWA

SEQ ID NO: 5 - protein, 8 aa, Artificial Sequence
  Description of Artificial Sequence Synthetic peptide
GPYAPAGH

SEQ ID NO: 6 - protein, 8 aa, Artificial Sequence
  Description of Artificial Sequence Synthetic peptide
GPNGILGN

SEQ ID NO: 7 - protein, 8 aa, Artificial Sequence
  Description of Artificial Sequence Synthetic peptide
GPNGIFGN

SEQ ID NO: 8 - protein, 5 aa, Artificial Sequence
  Description of Artificial Sequence Synthetic peptide
GPLGP

SEQ ID NO: 9 - protein, 8 aa, Artificial Sequence
  Description of Artificial Sequence Synthetic peptide
GPQGIAGN

SEQ ID NO: 10 - protein, 7 aa, Artificial Sequence
  Description of Artificial Sequence Synthetic peptide
GPLGVRG

SEQ ID NO: 11 - protein, 6 aa, Artificial Sequence
  Description of Artificial Sequence Synthetic peptide
PLAVGA

SEQ ID NO: 12 - protein, 4 aa, Artificial Sequence
  Description of Artificial Sequence Synthetic peptide
APGL

SEQ ID NO: 13 - protein, 7 aa, Artificial Sequence
  Description of Artificial Sequence Synthetic peptide
PQGIAGW

SEQ ID NO: 14 - protein, 8 aa, Artificial Sequence
  Description of Artificial Sequence Synthetic peptide
GPNGIAGN

SEQ ID NO: 15 - protein, 12 aa, Human immunodeficiency virus
CYGRKKRRQRRR

SEQ ID NO: 16 - protein, 13 aa, Human immunodeficiency virus
GRKKRRQRRRPPQ

SEQ ID NO: 17 - protein, 17 aa, Human immunodeficiency virus
TRQARRNRRRRWRERQR

SEQ ID NO: 18 - protein, 8 aa, Artificial Sequence
  Description of Artificial Sequence Synthetic peptide
RRRRRRRR

SEQ ID NO: 19 - protein, 12 aa, Artificial Sequence
  Description of Artificial Sequence Synthetic peptide
RRRRRRRRRRRR

SEQ ID NO: 20 - protein, 16 aa, Artificial Sequence
  Description of Artificial Sequence Synthetic peptide
RQIKIWFQNRRMKWKK

SEQ ID NO: 21 - protein, 18 aa, Artificial Sequence
  Description of Artificial Sequence Synthetic peptide
LLIILRRRIRKQAHAHSK

SEQ ID NO: 22 - protein, 19 aa, Artificial Sequence
  Description of Artificial Sequence Synthetic peptide
RQGAARVTSWLGLQLRIGK

SEQ ID NO: 23 - protein, 18 aa, Artificial Sequence
  Description of Artificial Sequence Synthetic peptide
RRLRRLLRRLRRLLRRLR

SEQ ID NO: 24 - protein, 12 aa, Artificial Sequence
  Description of Artificial Sequence Synthetic peptide
PRLPRLPRLPRL

SEQ ID NO: 25 - protein, 13 aa, Artificial Sequence
  Description of Artificial Sequence Synthetic peptide
GLSASPNLQFRTV

SEQ ID NO: 26 - protein, 4 aa, Artificial Sequence
  Description of Artificial Sequence Synthetic peptide
GPLG

SEQ ID NO: 27 - protein, 4 aa, Artificial Sequence
  Description of Artificial Sequence Synthetic peptide
IAGQ

SEQ ID NO: 28 - protein, 8 aa, Artificial Sequence
  Description of Artificial Sequence Synthetic peptide
GGGPALIQ

(19) United States
(12) Patent Application Publication
(10) Pub. No.: US 2015/0306241 A1
(43) Pub. Date: Oct. 29, 2015

(54) COPOLYMERS FOR THE DELIVERY OF DRUGS INTO CELLS

(71) Applicant: NORTHEASTERN UNIVERSITY, Boston, MA (US)

(72) Inventors: Lin Zhu, Malden, MA (US); Federico Perche, Boston, MA (US); Vladimir P Torchilin, Charlestown, MA (US)

(21) Appl. No.: 14/647,241

(22) PCT Filed: Nov. 27, 2013
(86) PCT No.: PCT/US13/72216

§ 371 Date: May 26, 2015

Related U.S. Application Data
(60) Provisional application No. 61/731,951, filed on Nov. 30, 2012.

(51) Int. Cl. A61K 47/48; A61K 31/337; C07K 7/08; C07K 7/06

(52) U.S. Cl. CPC A61K 47/48338; C07K 7/06; A61K 47/48215; A61K 31/337; A61K 47/48807; C07K 7/08

(57) ABSTRACT

Provided is a co-polymer of formula A-B-C or a pharmaceutically acceptable salt thereof, where A comprises a water soluble polymer; B comprises a matrix metalloprotease (MMP)-cleavable polypeptide; C is a chemotherapeutic drug or a derivative thereof; and A is connected to B at a first end through a first covalent bond or a first linking moiety and B is connected to C at a second end through a second covalent bond or a second linking moiety, where the co-polymer is not cross-linked.

### RELATED APPLICATION

The present application claims priority to U.S. provisional patent application U.S. Ser. No. 61/731,951 filed Nov. 30, 2012, which is incorporated herein by reference in its entirety.

### STATEMENT OF GOVERNMENT INTEREST

This invention was made with government support under RO1 CA121838 awarded by the National Institute of Health. The government has certain rights in the invention.

### BACKGROUND

Small molecule drugs having poor aqueous solubilities can be difficult to effectively administer to a patient. The intravenous (IV) administration of relatively water insoluble small molecule drugs requires large volumes of an aqueous vehicle and the subcutaneous delivery of such drugs can result in local toxicity and low levels of activity.

### SUMMARY

The present technology provides co-polymers, micellar compositions comprising the co-polymers, and related methods of use for the efficient delivery of a small-molecule drug to disease-associated target cells or tissues. In some embodiments, the disease is cancer. Delivery of the small-molecule drug is facilitated by the disclosed co-polymer which is covalently attached to the small-molecule drug and designed to release the drug at the disease-associated target cells or tissues.

According to one aspect, a co-polymer compound of formula A-B-C or a pharmaceutically acceptable salt thereof is provided, where A includes a water soluble polymer; B includes a matrix metalloprotease (MMP)-cleavable polypeptide; C is a chemotherapeutic drug or a derivative thereof; and A is connected to B at a first end through a first covalent bond or a first linking moiety and B is connected to C at a second end through a second covalent bond or a second linking moiety, where the co-polymer is not cross-linked.

According to another aspect, a micellar composition or mixed micellar composition is provided, where the micellar composition includes any of the co-polymers disclosed herein.

In yet another aspect, a method is provided for treating cancer in a subject including administering to the subject an effective amount of a composition including any of the co-polymers or micellar compositions disclosed herein.

In another aspect, a method is provided for delivering an chemotherapeutic drug into one or more cells of a subject including administering to the subject an effective amount of a composition including any of the co-polymers or the micellar compositions disclosed herein.

In some embodiments, the co-polymers, micellar compositions and mixed micellar compositions are found to (i) have higher micellization efficiency with low critical micelle concentration (CMC) and higher drug loading, (ii) impart higher stability and protection of the condensed siRNA against enzymatic degradation, (iii) have enhanced cell penetration resulting in efficient transfection, and (iv) have lesser cytotoxicity due to PEGylation and less systemic immunogenicity.

### BRIEF DESCRIPTION OF THE DRAWINGS

The foregoing and other objects, aspects, features and advantages of the disclosure will become more apparent and better understood by referring to the following description taken in conjunction with the accompanying non-limiting drawings.

FIGS. 1-3 illustrate a representative drug delivery strategy and characterization of the MMP2-sensitive nanopreparations.

FIG. 1 illustrates one potential mode of action for the co-polymers and micelles described herein.

FIG. 2 shows transmission electron microscopy (TEM) images. The particle size and morphology of the nanopreparations were analyzed by TEM using negative staining with 1% PTA.

FIG. 3 illustrates how enzymatic cleavage of the PEG2000-peptide-PTX was characterized. To determine the digestion of PEG2000-peptide-PTX (left) and its nanopreparation (right), the samples were treated with 5 ng/μL of MMP2 and run in chloroform/methanol (8:2, v/v) and visualized with Dragendorff's reagent.

FIGS. 4-9 illustrate the in vitro evaluation of a paclitaxel conjugate and its nanopreparations. The term “nanopreparation” as used herein is used interchangeably with “micellar composition”.

FIG. 4 illustrates the cytotoxicity of PEG2000-peptide-PTX in A549 and H9C2 cells. The cytotoxicity of monolayer cells was determined by Cell Titer-Blue® Cell Viability Assay after 72 h treatments.

FIG. 5 illustrates an apoptosis analysis. The apoptosis of A549 cells was determined by FACS using Annexin V/Propidium Iodide double staining after 72 h treatments.

FIG. 6 illustrates the cellular uptake in A549 cell monolayers. Cells were treated with NBD-PE-labeled formulations for 2 h before measurement. For FACS (left), cells were trypsinized and washed with PBS. For confocal microscopy (right), cells were fixed and stained with Hoechst 33342.

FIG. 7 illustrates the cytotoxicity of the nanopreparations in A549 cell monolayers. Cells were treated with moderate to low doses of PTX formulations for 72 h before Cell Titer-Blue® Cell Viability Assay.

FIG. 8 illustrates the penetration of the nanopreparations in A549 spheroids. The spheroids were treated with rhodamine-PE-labeled formulations for 2 h before confocal microscopy (a-h). The sections from c and d were stained by Hoechst 33342 (i and j); a, PEG1000-PE; b, TATp-PEG1000-PE; c and d, TATp-PEG1000-PE/PEG2000-peptide-PTX; e and f, PEG1000-PE/PEG2000-peptide-PTX; g and h, PEG2000-peptide-PTX; i and j, TATp-PEG1000-PE/PEG2000-peptide-PTX.

FIG. 9 illustrates the cytotoxicity of the nanopreparations in A549 spheroids. The spheroids were treated with PTX formulations at the dose of 29.5 ng/mL every other day for 6 days and the cytotoxicity was estimated by the LDH release.

FIGS. 10-13 illustrate the in vivo tumor targeting and antitumor efficacy of PEG2000-peptide-PTX.

FIG. 10 illustrates in vivo cell internalization data. HBSS, the rhodamine-labeled nanopreparation, and its non-sensitive counterpart were injected intravenously in tumor-bearing mice at 5 mg/Kg PTX, respectively. At 2 h post-injection, tumor and major organs (liver, kidney, spleen, heart, and lung) were collected. The cells were dissociated from fresh tissue and analyzed immediately by FACS.

FIG. 11 illustrates intratumor localization data. The tumor tissue sections were stained by Hoechst 33342 and detected by confocal microscopy.

FIG. 12 illustrates tumor growth inhibition (% of the starting tumor volume) data. Tumor size was measured every 3 days and calculated as V=lw²/2*, P<0.05 compared with other groups.

FIG. 13 illustrates tumor cell apoptosis data. Tumor sections were stained by Hoechst 33342, and apoptosis was analyzed by TUNEL assay under confocal microscopy.

FIGS. 14-17 illustrate the in vivo side toxicity assessment of PEG2000-peptide-PTX.

FIG. 14 illustrates mouse body weight (% of starting body weight) measurements.

FIG. 15 illustrates white blood cell counts. At the end of the experiment, white blood cells were counted by a hemocytometer.

FIG. 16 illustrates activity data for alanine transaminase (ALT) and aspartate transaminase (AST). The serum was separated from blood and the activity of transaminase was measured with ALT and AST assay kits.

FIG. 17 illustrates H&E staining experiments.

FIG. 18 illustrates characterizations of (A) PEG2000-peptide-PTX, (B) stability of PEG2000-peptide-PTX in plasma, and (C) TATp-PEG1000-PE by TLC. For characterization of PEG2000-peptide-PTX, the samples were run in chloroform/methanol (6:4, v/v) and visualized with Dragendorff's reagent (left) and UV 254 nm (right). For characterization of TATp-PEG1000-PE, the samples were run in chloroform/methanol (8:2, v/v) and visualized by Ninhydrin reagent (left), Dragendorff's reagent (middle) and Molybdenum blue (right).

FIG. 19 illustrates determinations of the critical micelle concentration (CMC). The CMC of the nanopreparations was determined by fluorescence spectroscopy using pyrene as a hydrophobic fluorescent probe. The samples were hydrated by HBSS (A-C) or HBSS containing 50% mouse serum (D) at a ten-fold serial dilution and incubated with shaking at room temperature for 24 h before measurement. The intensity ratio (1338/1334) was calculated and plotted against the logarithm of the micelle concentration. The CMC value was obtained as the crossover point of two tangents of the curves.

FIG. 20 illustrates MMP2 levels in cell culture media and mouse tissues. The same numbers of H9C2 and A549 cells were maintained in complete growth media for 3 days. The media was then collected and concentrated by ultrafiltration before (A) SDS-PAGE and (B) Zymography. For quantitative detection of MMP2, an MMP2 ELISA assay was performed to detect the MMP2 concentration in the original cell media (without concentration process) (C). To determine the MMP2 levels in tissues, tumors and major organs were collected and homogenized in PBS containing 0.5% Triton® X100. The homogenates were analyzed by the MMP2 ELISA assay and normalized by the concentration of the total protein (D).

FIG. 21 illustrates stability studies by the dynamic light scattering (DLS). Shown is stability data for the nanopreparations in HBSS for 0-4 h at 37° C. and for 3 weeks at 4° C. (A). To evaluate the serum stability, the nanopreparations were incubated with normal mouse sera (1:10, v/v) at 37° C. for 0-4 h (B). The percentage of the particles with the size >500 nm was determined and indicated on the histogram.

FIG. 22 illustrates in vitro drug release data. The PTX release was measured by RP-HPLC after dialysis (MWCO 2,000 Da) against 1M sodium salicylate for 24 h at 37° C. All samples were trypsinized at 37° C. for 1 h before measurement. (A) PTX/methanol/water mixture. Lower trace, the blank media containing trypsin; Middle trace, the outside media; Upper trace, the inside media. (B) TATp-PEG1000-PE/PEG2000-peptide-PTX micelles. Lower trace, the outside media (TATp-PEG1000-PE/PEG2000-peptide-PTX inside the tube); Middle trace, the outside media (TATp-PEG1000-PE/PEG2000-peptide-PTX+MMP2 overnight, inside the tube); Upper trace, the inside media (TATp-PEG1000-PE/PEG2000-peptide-PTX+MMP2 overnight, inside the tube).

FIG. 23 illustrates tubulin immunostaining data. A549 cells were seeded on glass coverslips and treated with 24 nM of PTX formulations at 37° C. overnight. Then, the cells were fixed and permeabilized, followed by staining with a mouse monoclonal anti-β-Tubulin antibody and a donkey anti-mouse IgG FITC conjugated antibody. Finally, the cell nuclei were visualized by Hoechst 33342 before confocal microscopy.

FIG. 24 illustrates TATp competition studies. A549 cells were seeded in 24-well plates at 1.6×10⁵ cells/well in 300 μL/well of complete growth media. After 24 h, the free TATp (0.35, 3.5 or 35 μM) was added into the cell media. Then, 10 μL of the rhodamine-PE-labeled MMP2-sensitive nanopreparation (1 mg/mL) was immediately added and incubated for 2 h. The cells were collected and analyzed by FACS.

FIG. 25 illustrates tissue distributions of PTX. At 2 h after i.v. injection of 5 mg/Kg PTX formulations, the tumor, blood and major organs were collected, weighed and homogenized in PBS. The homogenates were extracted with 10 volumes of t-butyl methyl ether followed by centrifugation. The extracted PTX was reconstituted by methanol and analyzed by RP-HPLC. The unit for blood is μg/mL. The unit for other tissues is μg/g.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined and designed in a wide variety of different configurations, all of which are explicitly contemplated and made part of this disclosure.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

In general, “substituted” refers to an alkyl or alkenyl or polyamino group, as defined below in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group will be substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, alkynoxy, aryloxy, aralkyloxy, heterocyclyloxy, and heterocyclylalkoxy groups; carbonyls (oxo); carboxyls; esters; urethanes; oximes; hydroxylamines; alkoxyamines; aralkoxyamines; thiols; sulfides; sulfoxides; sulfones; sulfonyls; sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; nitriles (i.e., CN); and the like.

As used herein, “alkyl” groups include straight chain and branched alkyl groups having from 1 to about 20 carbon atoms, and typically from 1 to 12 carbons or, in some embodiments, from 1 to 8 carbon atoms. As employed herein, “alkyl groups” include cycloalkyl groups as defined below. Unless expressly indicated otherwise, alkyl groups may be substituted, or unsubstituted, and if no designation is used, it is assumed that the alkyl group may be either substituted or unsubstituted. Examples of straight chain alkyl groups include methyl, ethyl, n-propyl, n-butyl, n-pentyl, n-hexyl, n-heptyl, and n-octyl groups. Examples of branched alkyl groups include, but are not limited to, isopropyl, sec-butyl, t-butyl, neopentyl, and isopentyl groups. Representative substituted alkyl groups may be substituted one or more times with, for example, amino, thio, hydroxy, cyano, alkoxy, and/or halo groups such as F, Cl, Br, and I groups. As used herein, the term haloalkyl is an alkyl group having one or more halo groups. In some embodiments, haloalkyl refers to a per-haloalkyl group. In some embodiments, alkyl refers to the alkyl side chain derived from lauric (C12), myristic (C14), palmitic (C16) or stearic (C18) acid.

Alkenyl groups are straight chain, branched or cyclic alkyl groups having 2 to about 20 carbon atoms, and further including at least one double bond. In some embodiments alkenyl groups have from 1-12 carbons or, typically, from 1-8 carbon atoms. Unless expressly indicated otherwise, alkenyl groups may be substituted or unsubstituted, and if no designation is used, it is assumed that the alkenyl group may be either substituted or unsubstituted. Alkenyl groups include, for instance, vinyl, propenyl, 2-butenyl, 3-butenyl, isobutenyl, cyclohexenyl, cyclopentenyl, cyclohexadienyl, butadienyl, pentadienyl, and hexadienyl groups among others. Alkenyl groups may be substituted similarly to alkyl groups. Divalent alkenyl groups, i.e., alkenyl groups with two points of attachment, include, but are not limited to, CH—CH═CH₂, C═CH₂, or C═CHCH₃. In some embodiments, the alkenyl group corresponds to the monounsaturated or polyunsaturated sidechain from palmitoleic (16:1 n-7), cis-vaccenic acid (18:1 n-7), oleic acid (18:1 n-9), linoleic acid (18:1 n-6), linoelaidic acid (18:1 n-3), arachidonic acid (20:4 n-6), eicosapentaenoic acid (20:5 n-3) or docosahexaenoic acid (22:6 n-3).

The terms “alkylene” and “alkenylene,” alone or as part of another substituent means a divalent radical derived from an alkyl or alkenyl group, respectively, as exemplified by —CH₂CH₂CH₂CH₂—. For alkylene and alkenylene linking groups, no orientation of the linking group is implied.

The term “amine” (or “amino”), as used herein, refers to —NHR and —NRR′ groups, where R, and R′ are independently hydrogen, or a substituted or unsubstituted alkyl, alkenyl, alkynyl, cycloalkyl, aryl or aralkyl group as defined herein. Examples of amino groups include —NH₂, methylamino, dimethylamino, ethylamino, diethylamino, propylamino, isopropylamino, phenylamino, benzylamino, and the like.

As used herein, “analog” or “derivative” refers to any variation of a chemotherapeutic drug that retains antineoplastic activity or any variation of a MMP-cleavable peptide that remains cleavable by MMP. As these terms are used in relation to a MMP-cleavable peptide, the analog or derivative of the MMP-cleavable peptide refers to a polypeptide that may be fragmented or mutated, but is still cleaved by a MMP.

“Cancer” refers to a broad group of disease involving unregulated cell growth and division. Non-limiting examples of cancers include leukemias, lymphomas, carcinomas, and other malignant tumors, including solid tumors, of potentially unlimited growth that can expand locally by invasion and systemically by metastasis. Examples of cancers include any of those described herein, but are not limited to, cancer of the adrenal gland, bone, brain, breast, bronchi, colon and/or rectum, gallbladder, head and neck, kidneys, larynx, liver, lung, neural tissue, pancreas, prostate, parathyroid, skin, stomach, and thyroid. Certain other examples of cancers include, acute and chronic lymphocytic and granulocytic tumors, adenocarcinoma, adenoma, basal cell carcinoma, cervical dysplasia and in situ carcinoma, Ewing's sarcoma, epidermoid carcinomas, giant cell tumor, glioblastoma multiforma, hairy-cell tumor, intestinal ganglioneuroma, hyperplastic corneal nerve tumor, islet cell carcinoma, Kaposi's sarcoma, leiomyoma, leukemias, lymphomas, malignant carcinoid, malignant melanomas, malignant hypercalcemia, marfanoid habitus tumor, medullary carcinoma, metastatic skin carcinoma, mucosal neuroma, myeloma, mycosis fungoides, neuroblastoma, osteo sarcoma, osteogenic and other sarcoma, ovarian tumor, pheochromocytoma, polycythermia vera, primary brain tumor, small-cell lung tumor, squamous cell carcinoma of both ulcerating and papillary type, hyperplasia, seminoma, soft tissue sarcoma, retinoblastoma, rhabdomyosarcoma, renal cell tumor, topical skin lesion, veticulum cell sarcoma, and Wilm's tumor.

“Patient” and “subject” are used interchangeably to refer to a mammal in need of treatment e.g., for cancer. Generally, the patient is a human. In some embodiments, the patient is a human diagnosed with cancer. In certain embodiments a “patient” or “subject” may refer to a non-human mammal used in screening, characterizing, and evaluating drugs and therapies, such as, a non-human primate, a dog, cat, rabbit, pig, mouse or a rat.

“Solid tumor” refers to solid tumors including, but not limited to, metastatic or non-metastatic tumors in bone, brain, liver, lungs, lymph node, pancreas, prostate, skin and soft tissue (sarcoma).

“Therapeutically effective amount” of a co-polymer or micelle refers to an amount of the co-polymer or micelle that, when administered to a patient with cancer, will have the intended therapeutic effect, e.g., alleviation, amelioration, palliation or elimination of one or more manifestations of cancer in the patient. A therapeutic effect does not necessarily occur by administration of one dose, and may occur only after administration of a series of doses. Thus, a therapeutically effective amount may be administered in one or more administrations.

“Administering” or “administration of” a co-polymer or micelle to a subject or patient (and grammatical equivalents of this phrase) refers to direct administration, which may be administration to a patient by a medical professional or may be self-administration, and/or indirect administration, which may be the act of prescribing a co-polymer or micelle. For example, a physician who instructs a patient to self-administer a co-polymer or micelle and/or provides a patient with a prescription for a co-polymer or micelle is administering the co-polymer or micelle to the patient.

“Treating,” “treatment of,” or “therapy of” a condition or patient refers to taking steps to obtain beneficial or desired results, including clinical results. Beneficial or desired clinical results include, but are not limited to, alleviation or amelioration of one or more symptoms of cancer; diminishment of extent of disease; delay or slowing of disease progression; amelioration, palliation, or stabilization of the disease state; or other beneficial results. Treatment of cancer may, in some cases, result in partial response or stable disease.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl (i.e., C₁-C₆) sulfonate and aryl sulfonate.

Many chemotherapeutic drugs, especially small molecule chemotherapeutic drugs, have poor aqueous solubility and are thus difficult to administer to a patient. The co-polymers disclosed herein include a water soluble polymer covalently bound to a matrix metalloprotease (MMP)-cleavable polypeptide that is itself covalently bound to a chemotherapeutic drug. In aqueous solution, the co-polymer self-assembles into micelles. The water soluble polymer confers aqueous solubility to the co-polymer and the attached chemotherapeutic drug. The water soluble polymer also allows the co-polymer to form micelles or mixed micelles that reduce the toxicity of chemotherapeutic drug to healthy cells while the chemotherapeutic drug is being transported to cancer cells. The matrix metalloprotease (MMP)-cleavable polypeptide allows the chemotherapeutic drug to remain covalently bound to the co-polymer during an administration of the co-polymer, until the co-polymer reaches cancerous cells and is cleaved by matrix metalloprotease, resulting in a fragment of the co-polymer that includes the drug covalently bound to a cleaved fragment of the MMP-cleavable polypeptide. Upon cleavage of chemotherapeutic drug from the cleaved fragment of the MMP-cleavable polypeptide by a protease such as trypsin, the chemotherapeutic drug can enter and kill cancer cells. See Scheme 1 below.

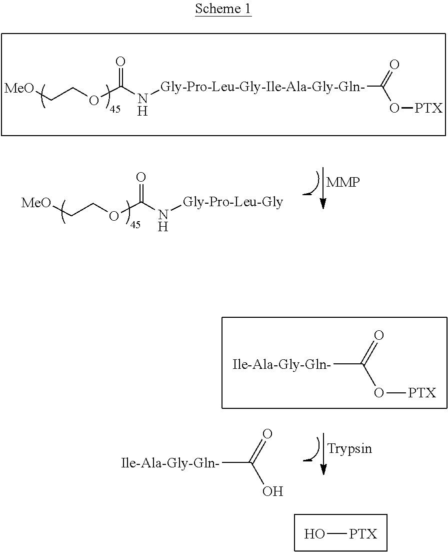

The terms “PTX” or “HO-PTX” both refer to paclitaxel which has two reactive hydroxyl substituents at the 2′ and 7′ positions, either of which can react to form an ester of the co-polymer in Scheme 1.

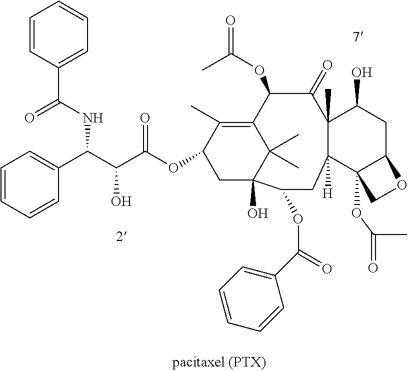 [SMILES: CC(=O)O[C@H]1C(=O)[C@@]2(C)[C@H](C[C@H](OC(=O)c3ccccc3)[C@]3(O)C[C@H](OC(=O)[C@H](O)[C@@H](NC(=O)c4ccccc4)c4ccccc4)C(C)=C1C3(C)C)[C@]1(OC(C)=O)CO[C@@H]1C[C@@H]2O]

Compared to conventional chemotherapeutic drugs, MMP-containing hydrophilic matrix or hydrogel prodrugs, administered alone or with known drug delivery systems, the co-polymers provide: (i) a high drug loading efficiency, (ii) a low risk of premature drug release or drug leakage, (iii) an enhanced tumor targeting, and (iv) an enhanced drug internalization by the tumor cells, (v) reduced toxicity to healthy cells.

The co-polymers and micelles described herein are not crosslinked, as is a crosslinked MMP-containing hydrophilic matrix or hydrogel prodrug. By specifying that the co-polymers are not “crosslinked” it is meant that two or more co-polymer compounds of formula I or II are covalently linked together. As such, in some embodiments, each co-polymer compound of formula I or II can have no more than a single drug covalently bound to the co-polymer compound. By contrast, a MMP-containing hydrophilic matrix or hydrogel prodrug is crosslinked together into a relatively rigid water-swellable network that includes numerous drug compounds, some of which are trapped within the internal core of hydrogel.

Because the co-polymers, micelles and mixed micelles do not form a crosslinked hydrophilic matrix or a hydrogel, the co-polymers, micelles and mixed micelles form a relatively dynamic drug delivery system. Upon administration, the co-polymers can combine to form micelles or mixed micelles that sequester the covalently bound drug moieties and reduce the toxicity of the drug. Upon reaching cancerous cells, the MMP-cleavable polypeptide portion of the co-polymers of the micelles or mixed micelles are cleaved by MMPs to release the covalently bound drug moieties and kill cancer cells. The remaining uncleaved co-polymers can recombine into further micelles or mixed micelles to repeat the cycle of drug delivery. By contrast a crosslinked MMP-containing hydrophilic matrix or hydrogel prodrug is static. MMPs can only access the exterior of the crosslinked matrix or hydrogel, but not the interior. Consequently, excessive drug loadings are needed for a crosslinked MMP-containing hydrophilic matrix or hydrogel prodrug relative to the co-polymers, micelles or mixed micelles described herein.

### Copolymers and Micelles

In one aspect, a co-polymer of formula (I) is provided having the formula A-B-C or a pharmaceutically acceptable salt thereof, where A includes a water soluble polymer; B includes a matrix metalloprotease (MMP)-cleavable polypeptide; C is a drug, such as a chemotherapeutic drug or a derivative thereof; and A is connected to B at a first end through a first covalent bond or a first linking moiety and B is connected to C at a second end through a second covalent bond or a second linking moiety, wherein the co-polymer is not cross-linked.

The water-soluble polymer, A, can include any water-soluble and non-toxic polymer such as, but not limited to, polyvinylpyrrolidone, polyoxazoline, polyacrylamide, polymorpholine polyvinyl alcohol, polyvinyl pyrrolidine, methylcellulose, ethyl cellulose, carboxymethyl cellulose, hydroxymethylcellulose, hydroxypropyl methylcellulose, microcrystalline cellulose, or a polyether such as polyglycerol or poly(ethylene glycol) “PEG” where the water-soluble polymer is optionally substituted.

As shown in the Examples below, the nature and size of the water-soluble polymer, such as PEG, on the co-polymer can be altered to tune the properties of micelles made from the co-polymer, such as the micelle particle size, morphology and critical micelle concentration. These properties of the micelle can in turn be optimized to improve the ability of the co-polymer to safely and effectively deliver its covalently bound drug to cancer cells in a subject.

In some embodiments, the co-polymer is of formula (II) A¹-L¹-B-C, or a pharmaceutically acceptable salt thereof, where A¹ is XO(R₁O)ₙ—; X is hydrogen, acyl or alkyl; L¹ is a first linking moiety; B includes a MMP-cleavable polypeptide; C is a drug, such as a chemotherapeutic drug or a derivative thereof; R₁ is C₂-C₈ alkylene, optionally substituted; and n is from 1 to 500. The term “acyl” refers to the substituent —CO-alkyl, such as —COCH₃.

In some embodiments, R₁ is C₂ alkylene, optionally substituted. In some embodiments, R₁ is C₃ alkylene, optionally substituted. In some embodiments, R₁ is C₄ alkylene, optionally substituted.

In some embodiments, R₂ is hydrogen. In some embodiments, R₂ is methyl. In some embodiments, R₂ is ethyl.

In some embodiments, X is hydrogen. In some embodiments, X is C₁-C₆ alkyl. For example, X may be methyl, ethyl, propyl or butyl. In some embodiments, X is acyl, such as CH₃CO— (i.e., “Ac”).

In some embodiments, A¹ is PEG or a derivative thereof. The polyether PEG moiety of A¹ of the co-polymer may have an average molecular weight ranging from about 100 daltons to about 20,000 daltons. In certain instances, the PEG moiety has an average molecular weight of from about 100 daltons to about 15,000 daltons. In some embodiments, the PEG moiety has an average molecular weight of about 1,500 daltons to about 5,000 daltons. In some embodiments, the polyether PEG moiety is PEG100, PEG200, PEG300, PEG400, PEG500, PEG600, PEG700, PEG800, PEG900, PEG1000, PEG2000, PEG3000, PEG 5000 or PEG10,000, i.e., PEG polyethers having an average molecular weight of approximately 100, 200, 300, 400, 500, 600, 700, 800, 900, 1000, 2,000, 3,000, 5,000 or 10,000 Daltons, respectively.

In some embodiments, A¹ is XO(CH₂CH₂O)ₙ— and n is from 1 to 500. In some embodiments, n is from 1 to 5, 6 to 10, 11 to 20, 21 to 30, 31 to 40, 41 to 50, 51 to 60, 61 to 70, 71 to 80, 81 to 90, 91 to 100, 101 to 200, 201 to 300, 301 to 400, 401 to 500.

In some embodiments, L¹ is —CO—, —CH₂CH₂CO— or —SO₂—. In some embodiments, L¹ is —CO—.

Substituent B includes any MMP-cleavable polypeptide. As used herein, “matrix metalloproteinases” (MMP's) are a class of extracellular enzymes including collagenase, stromelysin, and gelatinase which are believed to be involved in tissue destruction which accompanies a large number of disease states varying from arthritis to cancer. This group of enzymes with different substrate specificity contributes to the degradation of extracellular matrix comprising such complex components as collagen, proteoglycan, elastin, fibronectin, and laminin. In particular, MMP cleavage of the ECM protein facilitates cellular invasion and migration.

MMPs include interstitial collagenase (MMP-1), 72 kDa gelatinase (also known as type IV collagenase or gelatinase A; MMP-2), 92 kDa gelatinase (also known as type IV collagenase or gelatinase B; MMP-9), stromelysin-1 (MMP-3), matrilysin (MMP-7), neutrophil collagenase (MMP-8), stromelysin-2 (MMP10), stromelysin-3 (MMP-11), metalloelastase (MMP12), the MT-MMPs (MMP14, MMP15, MMP16, MMP17) and enamelysin (MMP19). With the exception of MMP-7, the primary structure among the family of reported MMPs comprises essentially an N-terminal propeptide domain, a Zn⁺⁺ binding catalytic domain and a C-terminal hemopexin-like domain. In MMP-7 there is no hemopexin-like domain. MMP-2 and MMP-9 contain an additional gelatin-binding domain. In addition, a proline-rich domain highly homologous to a type V collagen alpha 2 chain is inserted in MMP-9 between the Zn⁺⁺ binding catalytic domain and the C-terminal hemopexin-like domain.

In some embodiments, the MMP-cleavable polypeptide is MMP-1, MMP-2, MMP-3, MMP-7, MMP-8, MMP-9, MMP-10, or MMP-11. In some embodiments, the MMP-cleavable polypeptide is a MMP2-cleavable polypeptide. In some embodiments, the MMP-cleavable polypeptide is a MMP9-cleavable polypeptide.

MMPs play a role in normal and pathological processes, including embryogenesis, wound healing, inflammation, restenosis, arthritis, apoptosis and cancer. MMPs are known to be involved and over-expressed in many stages of human tumors including breast cancer, colorectal cancer, lung cancer, liver cancer, prostate cancer, pancreatic cancer, and ovarian cancer (Atkinson J M, et al. Cancer research (2010) 70(17):6902-6912 and Nguyen Q T, et al. Proc Natl Acad Sci USA (2010) 107(9):4317-4322.) In highly metastatic tumor cells, there are reports of conspicuous expression of type IV collagenase (MMP-2, MMP-9) which mainly degrade type IV collagen (Cancer Res., 46:1-7, 1986; Biochem. Biophys. Res. Commun., 154:832-838, 1988; Cancer, 71:1368-1383, 1993).

The metastasis of tumor cells progresses via destruction of basement membranes, invasion into and effusion from blood vessels, successful implantation on secondary organs, further growth and the like. The extracellular matrix that blocks tumor metastasis is composed of various complex components, including type IV collagen, proteoglycans, elastin, fibronectin, laminin, heparan sulfate, and the like. And these matrix metalloproteinases, with their distinct substrate specificities are responsible for the degradation of the extracellular matrix. Among these MMPs, it has been reported that type IV collagenase (MMP-2 and MMP-9) is highly expressed in high metastatic tumor cells. Thus, in some embodiments, the MMP-sensitive peptide is meant to be a MMP-2 and/or MMP-9 sensitive peptide.

As used herein, “MMP cleavable polypeptide” refers to that polypeptide having an amino acid sequence, which is recognized and cleaved by at least one of the MMPs. As such, the MMP-cleavable polypeptide is a peptide of any sequence that is cleaved by MMP relative to a control peptide that is not cleaved by MMP. The recognition may be specific to a particular MMP or it may be general to all MMPs. An example of a polypeptide that is cleaved by an MMP is collagen, gelatin, elastin and silk-elastin. However, it is understood that the polypeptide sequence need not be a full-length protein such as collagen or elastin. It is understood that any fragment or derivative of these MMP substrates that is cleaved by an MMP falls within the purview of the term “MMP cleavable polypeptide.” Furthermore, it is understood that the MMP cleavable polypeptide encompasses polypeptides that are at least partly synthetic and/or chimeric, such as silk-elastin, and fragments thereof.

MMPs cleave primarily at Leu-Gly or Ile-Gly bonds. Thus, in some embodiments, the MMP cleavable polypeptide is a peptide comprising Leu-Gly dipeptide sequence. In other embodiments, the MMP cleavable polypeptide is a peptide comprising Ile-Gly dipeptide sequence. In some embodiments, the MMP cleavable polypeptide is a polypeptide of 5 to 30 amino acid residues. In some embodiments, the MMP cleavable polypeptide is a polypeptide of 5 to 15 amino acid residues.

In some embodiments, the MMP-cleavable polypeptide is -Gly-Pro-Leu-Gly-Ile-Ala-Gly-Gln- (SEQ ID NO:1); -Pro-Leu-Gly-Leu-Trp-Ala- (SEQ ID NO:2); -Pro-Leu-Gly-Leu-Gly-Ala- (SEQ ID NO:3); -Pro-Leu-Gly-Leu-Trp-Ala- (SEQ ID NO:4); -Gly-Pro-Tyr-Ala-Pro-Ala-Gly-His- (SEQ ID NO:5); -Gly-Pro-Asn-Gly-Ile-Leu-Gly-Asn- (SEQ ID NO:6); -Gly-Pro-Asn-Gly-Ile-Phe-Gly-Asn- (SEQ ID NO:7); -Gly-Pro-Leu-Gly-Pro- (SEQ ID NO:8); -Gly-Pro-Gln-Gly-Ile-Ala-Gly-Asn- (SEQ ID NO:9); -Gly-Pro-Leu-Gly-Val-Arg-Gly- (SEQ ID NO:10); -Pro-Leu-Ala-Nva-Gly-Ala- (SEQ ID NO:11); -Ala-Pro-Gly-Leu- (SEQ ID NO:12); -Pro-Gln-Gly-Ile-Ala-Gly-Trp- (SEQ ID NO:13); or -Gly-Pro-Asn-Gly-Ile-Ala-Gly-Asn- (SEQ ID NO:14). In some embodiments, the MMP-cleavable polypeptide is —NH-Gly-Pro-Leu-Gly-Ile-Ala-Gly-Gln-CO₂— (SEQ ID NO:1).

Substituent C includes any small-molecule drug, such as a chemotherapeutic drug, or a derivative thereof, that is covalently bound to substituent B, either directly via a covalent bond or indirectly via a second linker moiety L². In some embodiments the second linking moiety, L², is —CO—, —COO—, —CONR₂—, —CH₂CH₂CO—, —CH₂CH₂COO—, —CH₂CH₂CONR₂—, —SO₂— or —SO₂NR₂—. In some embodiments, the small-molecule drug includes, but is not limited to, a chemotherapeutic drug, although the present technology is not limited by the nature of the small-molecule or chemotherapeutic drug.

Useful chemotherapeutic drugs are selected from those drugs that impede or block tumorigenesis, angiogenesis, cell proliferation, or by way of example, but not by way of limitation, anti-apoptosis in the breast tissue of a subject, e.g., mammal. In one embodiment, the chemotherapeutic drug impedes or blocks the activity of a peptide or protein whose activity promotes tumorigenesis, angiogenesis, cell proliferation, or anti-apoptosis in the breast tissue of the mammal. For example, it may impede or block the activity of a peptide or protein that causes or promotes the growth of a breast cancer or causes or promotes its metastasis. In one embodiment, it impedes or blocks the activity of a protein that is a pro-tumorigenic pathway protein, a pro-angiogenesis pathway protein, a pro-cell proliferation pathway protein, or an anti-apoptotic pathway protein. Such proteins include, but are not limited to, an EGFR pathway protein, Raf-1 pathway protein, mTOR pathway protein, VEGF pathway protein, HIF-1 alpha pathway protein, Her-2 pathway protein, PDGF pathway protein, or Cox-2 pathway protein. Particular examples of proteins that may be targeted by the therapeutic agent are: EGFR, Raf-1, mTOR, VEGF, HIF-1 alpha, Her-2, PDGF, or Cox-2.

Suitable chemotherapeutic drugs are described in REMINGTON'S PHARMACEUTICAL SCIENCES, 19th Ed. (Mack Publishing Co. 1995), and in GOODMAN AND GILMAN'S THE PHARMACOLOGICAL BASIS OF THERAPEUTICS, 7th Ed. (MacMillan Publishing Co. 1985), as well as revised editions of these publications. Other suitable chemotherapeutic agents, such as experimental drugs, are known to those of skill in the art.

The small-molecule drug, such as a chemotherapeutic drug, may include a free hydroxyl group, free primary or secondary amino group or both hydroxyl and amino groups. Chemotherapeutic drugs having a free hydroxyl group can be incorporated into the co-polymers as esters. Chemotherapeutic drugs having a free amino groups can be incorporated into the co-polymers as amides. Chemotherapeutic drugs having a free alcohol & amino groups can be incorporated into the co-polymers as esters and/or amides.

For example, the chemotherapeutic drugs may be a hydroxy-containing chemotherapeutic drug such as Aclacinomycins, Arzoxifene, Batimastat, Broxuridine, Calusterone, Capecitabine, CC-1065, Chromomycins, Diethylstilbestrol, Docetaxel, Doxifluridine, Droloxifene, Dromostanolone, Enocitabine, Epitiostanol, Estramustine, Etanidazole, Etoposide, Fenretinide, Flavopiridol, Formestane, Fosfestrol, Fulvestrant, Gemcitabine, Irinotecan, Melengestrol, Menogaril, Miltefosine, Mitobronitol, Mitolactol, Mopidamol, Nitracrine, Nogalamycin, Nordihydroguaiaretic Acid, Olivomycins, Paclitaxel and other known paclitaxel analogs, Plicamycin, Podophyllotoxin, Retinoic acid, Roquinimex, Rubitecan, Seocalcitol, Temoporfin, Teniposide, Tenuazonic Acid, Topotecan, Valrubicin, Vinblastine, Vincristine or Zosuquidar. In some embodiments, the chemotherapeutic drug is Paclitaxel or a Paclitaxel analog. Paclitaxel is one of the most commonly used antineoplastic agents for the treatment of solid tumors including ovarian, breast, non-small cell lung, head and neck cancers. However, its clinical application is complicated by its low water-solubility, off-target toxicity and acquired drug resistance.

The chemotherapeutic drugs may also be an amino-containing chemotherapeutic drug such as 9-Aminocamptothecin, Aminolevulinic Acid, Amsacrine, Bisantrene, Cactinomycin, Carboquone, Carmofur, Carmustine, Cyclophosphamide, Dacarbazine, Dactinomycin, Demecolcine, Diaziquone, 6-Diazo-5-oxo-L-norleucine (DON), Edatrexate, Efaproxiral, Eflornithine, Eniluracil, Erlotinib, Fluorouracil, Gefitinib, Gemcitabine, Goserelin, Histamine, Ifosfamide, Imatinib, Improsulfan, Lanreotide, Leuprolide, Liarozole, Lobaplatin, Cisplatin, Carboplatin, Lomustine, Lonafarnib, Mannomustine, Melphalan, Methotrexate, Methyl Aminolevulinate, Miboplatin, Mitoguazone, Mitoxantrone, Nilutamide, Nimustine, Nolatrexed, Oxaliplatin, Pemetrexed, Phenamet, Piritrexim, Procarbazine, Raltitrexed, Tariquidar, Temozolomide, Thiamiprine, Thioguanine, Tipifamib, Tirapazamine, 3-Aminopyridine-2-carboxaldehyde thiosemicarbazone, 3-Aminopyridine-4-methyl-2-carboxaldehyde thiosemicarbazone, Trimetrexate, Uracil Mustard, Uredepa or Meturedepa.

The chemotherapeutic drugs may be a hydroxy- and amino-containing chemotherapeutic drug such as Ancitabine, Anthramycin, Azacitidine, Bleomycins, Bropirimine, Buserelin, Carubicin, Chlorozotocin, Cladribine, Cytarabine, Daunorubicin, Decitabine, Defosfamide, Docetaxel, Doxorubicin, Ecteinascidins, Epirubicin, Gemcitabine, Hydroxyurea, Idarubicin, Marimastat, 6-Mercaptopurine, Pentostatin, Peplomycin, Perfosfamide, Pirarubicin, Prinomastat, Puromycin, Ranimustine, Streptonigrin, Streptozocin, Tiazofurin, Troxacitabine, Vindesine or Zorubicin. In some embodiments, the chemotherapeutic drug is doxorubicin.

In one embodiment, the co-polymer of formula (I) has the structure:

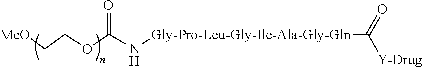

where n is from 2 to 100, Y is —O— or —NH— and “drug” is a covalently bound chemotherapeutic drug. In some embodiments, n is 10-30. In some embodiments, n is 31-50.

In one embodiment, the co-polymer of formula (I) is “PEG2000-peptide-PTX” having the structure:

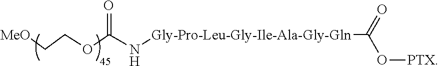

In one embodiment, the co-polymer of formula (I) is “PEG1000-peptide-PTX” having the structure:

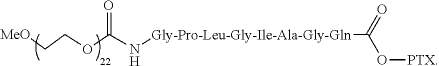

For the representative co-polymers PEG1000-peptide-PTX and PEG2000-peptide-PTX (the synthesis of which is shown below in Scheme 2), PEG1000 is CH₃(CH₂CH₂O)₂₂—, PEG2000 is CH₃(CH₂CH₂O)₄₅—, the peptide is —NH-Gly-Pro-Leu-Gly-Ile-Ala-Gly-Gln-CO₂— (SEQ ID NO:1), PTX is paclitaxel, where PEG1000 or PEG2000 and the peptide are joined via a —CO— linker and PTX is joined to the peptide by an ether bond via the 2′ hydroxyl or the 7′ hydroxyl of PTX.

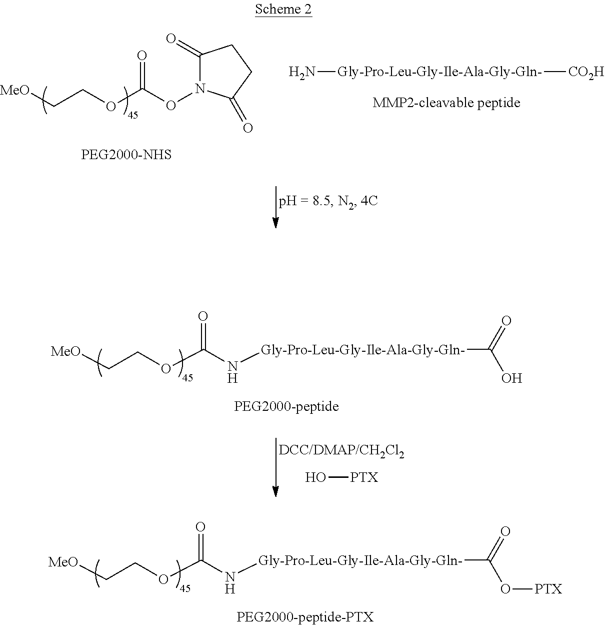

In another aspect, provided is a micellar composition that includes any of the co-polymers disclosed herein and water. In some embodiments, the micellar composition includes more than one co-polymer, such as co-polymers having different-sized PEG groups. For example, mixed micelles can be prepared from mixtures of PEG1000-peptide-PTX and PEG2000-peptide-PTX. As shown in the Examples below, the micelle particle size, morphology and critical micelle concentration can be tuned by altering the size of the water-soluble polymer, such as PEG, on the co-polymer.

In aqueous solution, the co-polymers spontaneously form micelles. The micellar composition, that includes any of the co-polymers disclosed herein, may further include an amphiphilic compound, other than the co-polymer, to form mixed micelles. The term “amphiphilic compound” as used herein refers to any amphiphilic compound other than the co-polymer that can be combined with one or more co-polymers to form mixed-micelles. Representative amphiphilic compounds include lipids or phospholipids, for example, such as any of those described below.

In some embodiments, the amphiphilic compound is of formula (III), D-E-F, where D includes a hydrophobic moiety, optionally substituted, E is a water-soluble polymer, optionally substituted, F is absent or F is hydrogen, alkyl, acyl or a polypeptide, optionally substituted. D and E are joined directly by a third covalent bond or indirectly by a third linking moiety, L³, and E and F are joined directly by a fourth covalent bond or indirectly by a fourth linking moiety, L⁴.

As noted, the amphiphilic compound of formula (III), D-E-F, includes a hydrophobic moiety D. For example, the hydrophobic moiety D may include —C₆-C₂₂-alkyl or —C₆-C₂₂-alkenyl. In some embodiments, the hydrophobic moiety D derives from a saturated fatty acid, monounsaturated fatty acid or polyunsaturated fatty acid. In some embodiments, the saturated fatty acid is lauric (C12), myristic (C14), palmitic (C16) or stearic (C18) acid. In some embodiments, the monounsaturated fatty acid is palmitoleic (16:1 n-7), cis-vaccenic acid (18:1 n-7) or oleic acid (18:1 n-9). In some embodiments, the monounsaturated fatty acid is oleic acid. In some embodiments, the polyunsaturated fatty acid is linoleic acid (18:1 n-6), linoelaidic acid (18:1 n-3), arachidonic acid (20:4 n-6), eicosapentaenoic acid (20:5 n-3) and docosahexaenoic acid (22:6 n-3). The hydrophobic moiety D may also include the corresponding fatty amine or fatty alcohol derivative.

In some embodiments of the amphiphilic compound of formula (III) D-E-F, substituent D includes a phospholipid, optionally substituted. Phospholipids are amphiphilic compounds which typically contain at least one phosphate group and at least one, preferably two, lipophilic long-chain hydrocarbon group.

Examples of suitable phospholipids include esters of glycerol with one or preferably two (equal or different) residues of fatty adds and with phosphoric acid, wherein the phosphoric acid residue is in turn bound to a hydrophilic group, such as, for instance, choline (phosphatidylcholines—PC), serine (phosphatidylserines—PS), glycerol (phosphatidylglycerols—PG), ethanolamine (phosphatidylethanolamines—PE), inositol (phosphatidylinositol). Esters of phospholipids with only one residue of fatty acid are generally referred to in the art as the “lyso” forms of the phospholipid or “lysophospholipids”. Fatty acids residues present in the phospholipids are in general long chain aliphatic acids, typically containing from 12 to 24 carbon atoms, preferably from 14 to 22; the aliphatic chain may contain one or more unsaturations or is preferably completely saturated. Examples of suitable fatty acids included in the phospholipids are, for instance, lauric acid, myristic acid, palmitic acid, stearic acid, arachidic acid, behenic acid, oleic acid, linoleic acid, and linolenic acid. Preferably, saturated fatty acids such as myristic acid, palmitic acid, stearic acid and arachidic add are employed.

Further examples of phospholipid are phosphatidic acids, i.e., the diesters of glycerol-phosphoric acid with fatty acids; sphingolipids such as sphingomyelins, i.e., those phosphatidylcholine analogs where the residue of glycerol diester with fatty acids is replaced by a ceramide chain; cardiolipins, i.e., the esters of 1,3-diphosphatidylglycerol with a fatty acid; glycolipids such as gangliosides GM1 (or GM2) or cerebrosides; glucolipids; sulfatides and glycosphingolipids.

As used herein, the term phospholipids include either naturally occurring, semisynthetic or synthetically prepared products that can be employed either singularly or as mixtures. Examples of naturally occurring phospholipids are natural lecithins (phosphatidylcholine (PC) derivatives) such as, typically, soya bean or egg yolk lecithins.

Examples of semisynthetic phospholipids are the partially or fully hydrogenated derivatives of the naturally occurring lecithins. Preferred phospholipids are fatty acids di-esters of phosphatidylcholine, ethylphosphatidylcholine, phosphatidylglycerol, phosphatidic acid, phosphatidylethanolamine, phosphatidylserine or of sphingomyelin. Examples of preferred phospholipids are, for instance, dilauroyl-phosphatidylcholine (DLPC), dimyristoyl-phosphatidylcholine (DMPC), dipalmitoyl-phosphatidylcholine (DPPC), diarachidoyl-phosphatidylcholine (DAPC), distearoyl-phosphatidylcholine (DSPC), dioleoyl-phosphatidylcholine (DOPC), 1,2Distearoyl-sn-glycero-3-Ethylphosphocholine (Ethyl-DSPC), dipentadecanoyl-phosphatidylcholine (DPDPC), 1-myristoyl-2-palmitoyl-phosphatidylcholine (MPPC), 1-palmitoyl-2-myristoyl-phosphatidylcholine (PMPC), 1-palmitoyl-2-stearoyl-phosphatidylcholine (PSPC), 1-stearoyl-2-palmitoyl-phosphatidylcholine (SPPC), 1-palmitoyl-2-oleylphosphatidylcholine (POPC), 1-oleyl-2-palmitoyl-phosphatidylcholine (OPPC), dilauroylphosphatidylglycerol (DLPG) and its alkali metal salts, diarachidoylphosphatidylglycerol (DAPG) and its alkali metal salts, dimyristoylphosphatidylglycerol (DMPG) and its alkali metal salts, dipalmitoylphosphatidylglycerol (DPPG) and its alkali metal salts, distearoylphosphatidylglycerol (DSPG) and its alkali metal salts, dioleoyl-phosphatidylglycerol (DOPG) and its alkali metal salts, dimyristoyl phosphatidic acid (DMPA) and its alkali metal salts, dipalmitoyl phosphatidic acid (DPPA) and its alkali metal salts, distearoyl phosphatidic acid (DSPA), diarachidoylphosphatidic acid (DAPA) and its alkali metal salts, dimyristoylphosphatidylethanolamine (DMPE), dipalmitoylphosphatidylethanolamine (DPPE), distearoyl phosphatidyl-ethanolamine (DSPE), dioleylphosphatidylethanolamine (DOPE), diarachidoylphosphatidylethanolamine (DAPE), dilinoleylphosphatidylethanolamine (DLPE), dimyristoyl phosphatidylserine (DMPS), diarachidoyl phosphatidylserine (DAPS), dipalmitoyl phosphatidylserine (DPPS), distearoylphosphatidylserine (DSPS), dioleoylphosphatidylserine (DOPS), dipalmitoyl sphingomyelin (DPSP), and distearoylsphingomyelin (DSSP), dilauroyl-phosphatidylinositol (DLPI), diarachidoylphosphatidylinositol (DAPI), dimyristoylphosphatidylinositol (DMPI), dipalmitoylphosphatidylinositol (DPPI), distearoylphosphatidylinositol (DSPI), dioleoyl-phosphatidylinositol (DOPI).

In some embodiments, the phospholipid is dioleoylphosphatidyl ethanolamine (DOPE) phosphatidylethanolamine (cephalin) (PE), phosphatidic acid (PA), phosphatidylcholine (PC), 1,2-distearoyl-sn-glycero-3-phosphoethanolamine (DSPE) or phosphatidylserine (PS). In some embodiments, the phospholipid is DOPE.

Groups D and E may joined directly by a third covalent bond. Alternatively, groups D and E may be joined indirectly by a third linking moiety, L³, such as, for example, an amino group, —CO—, —CH₂CH₂CO—, —SO₂—, —NR²CO—, —NR²CH₂CH₂CO— or —NR²SO₂—, where R² is hydrogen or alkyl. Thus, in some embodiments, the amphiphilic compound has the formula D-L³-E-F.

The amphiphilic compound of formula (III), D-E-F, includes a water-soluble polymer E. The water-soluble polymer E may include, for example, any of the water-soluble polymers that are described above with regard to the co-polymer. Thus, the hydrophilic moiety E can include any water-soluble and non-toxic polymer such as, but not limited to, polyvinylpyrrolidone, polyoxazoline, polyacrylamide, polymorpholine polyvinyl alcohol, polyvinyl pyrrolidine, methylcellulose, ethyl cellulose, carboxymethyl cellulose, hydroxymethylcellulose, hydroxypropyl methylcellulose, microcrystalline cellulose, or a polyether such as polyglycerol or poly(ethylene glycol) “PEG” where the water-soluble polymer is optionally substituted. In some embodiments, water-soluble polymer E is interrupted with one or more linker groups such as —OCO—, —OCH₂CH₂CO— or —OSO₂—.

In some embodiments, E includes PEG or a derivative thereof. The polyether PEG moiety of A¹ of the co-polymer may have an average molecular weight ranging from about 100 daltons to about 20,000 daltons. In certain instances, the PEG moiety has an average molecular weight of from about 100 daltons to about 15,000 daltons. In some embodiments, the PEG moiety has an average molecular weight of about 1,500 daltons to about 5,000 daltons. In some embodiments, the polyether PEG moiety is PEG100, PEG200, PEG300, PEG400, PEG500, PEG600, PEG700, PEG800, PEG900, PEG1000, PEG2000, PEG3000, PEG 5000 or PEG10,000, i.e., PEG polyethers having an average molecular weight of approximately 100, 200, 300, 400, 500, 600, 700, 800, 900, 1000, 2,000, 3,000, 5,000 or 10,000 Daltons, respectively.

In some embodiments, E is —(OCH₂CH₂)—ₙ or —(OCH₂CH₂)ₙO— and n is from 1 to 500. In some embodiments, n is from 1 to 5, 6 to 10, 11 to 20, 21 to 30, 31 to 40, 41 to 50, 51 to 60, 61 to 70, 71 to 80, 81 to 90, 91 to 100, 101 to 200, 201 to 300, 301 to 400, 401 to 500.

The amphiphilic compound of formula (III), D-E-F, includes moiety F. In some embodiments, moiety F is hydrogen. In some embodiments, moiety F is —OH. In some embodiments, moiety F is C₁-C₆ alkyl, such as methyl, ethyl, propyl or butyl. In some embodiments, moiety F is —OC₁-C₆ alkyl. In some embodiments, moiety F is acyl, such as —COCH₃. In some embodiments, moiety F is O-acyl, such as —OCOCH₃.

In some embodiments, moiety F is polypeptide. For example, the polypeptide of moiety F may be a “cell penetrating peptide.” Cell penetrating peptides are functional carrier peptides twenty amino acid residues or less that are efficiently internalized into cells. Examples of known cell penetrating peptide classes include the amphipathic helical peptides and arginine-rich peptides. Illustrative cell penetrating peptides include transportan, model amphipathic peptide, the HIV cell-penetrating TAT peptide (TATp), artificial oligoarginine peptide, Antp or penetratin. (See the cell penetrating peptides described in Foged C, Nielsen H M. Exp Opin Drug Deliv. 2008; 5:105-17; and Hallbrink M, et al., Biochim Biophys Acta. 2001; 1515:101-9, all of which cell penetrating peptides are incorporated herein by reference.). For example, the HIV cell-penetrating TAT peptide (TATp) can have the peptide sequence Cys-Tyr-Gly-Arg-Lys-Lys-Arg-Arg-Gln-Arg-Arg-Arg (TATp, SEQ ID NO:15) or substitutions, deletions or modifications thereof.

In some embodiments, the cell penetrating peptide is a polypeptide of 5 to 30 amino acid residues. In some embodiments, the cell penetrating peptide is a polypeptide of 5 to 15 amino acid residues. In some embodiments, the cell penetrating peptide is an illustrative peptide listed in Table 1. In some embodiments, the cell penetrating peptide is -Cys-Tyr-Gly-Arg-Lys-Lys-Arg-Arg-Gln-Arg-Arg-Arg (SEQ ID NO:15).

TABLE 1
Illustrative Cell Penetrating Peptides

| | | SEQ |
|---|---|---|
| | | ID |
| Peptide | Sequence | NO: |
| Arginine-rich peptides | | |
| HIV-1 TATp | CYGRKKRRQRRR | 15 |
| HIV-1 Tat (48-60) | GRKKRRQRRRPPQ | 16 |
| HIV-1 Rev (34-50) | TRQARRNRRRRWRERQR | 17 |
| Arginine octamer (R8) | RRRRRRRR | 18 |
| Arginine dodecamer (R12) | RRRRRRRRRRRR | 19 |
| Amphipathic peptides | | |
| Penetratin | RQIKIWFQNRRMKWKK | 20 |
| pVEC | LLIILRRRIRKQAHAHSK | 21 |
| Erns | RQGAARVTSWLGLQLRIGK | 22 |
| RRL helix | RRLRRLLRRLRRLLRRLR | 23 |
| PRL4 | PRLPRLPRLPRL | 24 |
| Random | | |
| composition peptides | | |
| Random peptide | GLSASPNLQFRTV | 25 |

Groups E and F may joined directly by a fourth covalent bond. Alternatively, groups D and E may be joined indirectly by a fourth linking moiety, L⁴, such as, for example, —CO—, NR₂CO—, OCO—, —COO—, —CONR₂—, —CH₂CH₂CO—, —CH₂CH₂COO—, —CH₂CH₂CONR₂—, —SO₂—, NR₂SO₂—, or —SO₂NR₂—. The fourth linking moiety, L⁴, may derive from an acrylate, acrylamide or maleimide moiety. For example, the fourth linking moiety, L⁴, may be —NR₂COCH₂CH—, —OCOCH₂CH—, —COCH₂CH—, —NR₂CO(R^{A})COCH₂CH—, —OCO(R^{A})COCH₂CH—, —CO(R^{A})COCH₂CH—, NR₂CO(R^{A})R^{B}, —OCO(R^{A})R^{B}, —CO(R^{A})R^{B}, where R^{A} is —(CH₂)_{q}—, q is from 2 to 20, and R^{B} is

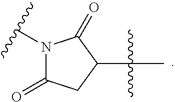 [SMILES: *C1CC(=O)N(*)C1=O]

In some embodiments, q is 2 to 4. In some embodiments, the fourth linking moiety, L⁴, is

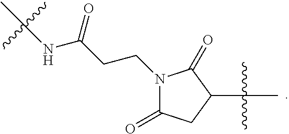 [SMILES: *NC(=O)CCN1C(=O)CC(*)C1=O]

Thus, in some embodiments, the amphiphilic compound has the formula D-E-L⁴-F. In some embodiments, the amphiphilic compound has the formula D-L³-E-L⁴-F.

In some embodiments, the amphiphilic compound has the formula D-L³-E-L⁴-F, where D is DOPE, PE, PA, PC, DSPE or PS; L³ is —CO—; E is —(OCH₂CH₂)—ₙ; n is from 10 to 50; and L⁴ is

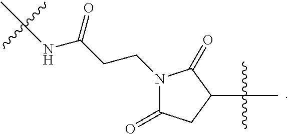 [SMILES: *NC(=O)CCN1C(=O)CC(*)C1=O]

In some embodiments, the amphiphilic compound is “PEG1000-DOPE” having the following structure:

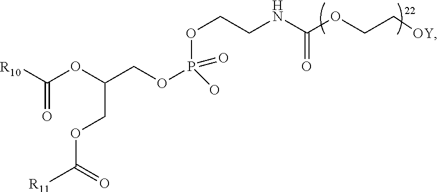

where R10 and R11 are Y is hydrogen or methyl.

In some embodiments, the amphiphilic compound is “TATp-PEG1000-DOPE” having the following structure:

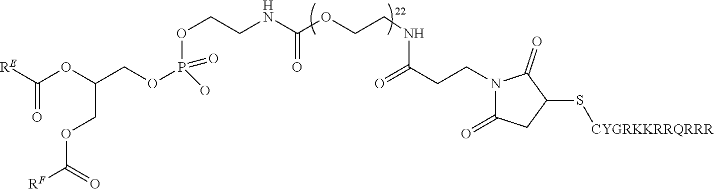

Synthesis of the co-polymer “TATp-PEG1000-DOPE” is shown below in Scheme 3, where TATp is CYGRKKRRQRRR, PEG1000 is —(OCH₂CH₂)₂₂—, DOPE is dioleoylphosphatidyl ethanolamine, where TATp, via its sidechain thiol at cysteine, and PEG1000 are joined via a

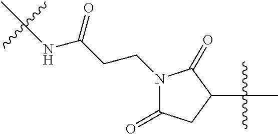 [SMILES: *NC(=O)CCN1C(=O)CC(*)C1=O]

linker, and PEG1000 and DOPE are joined via a —CO— linker.

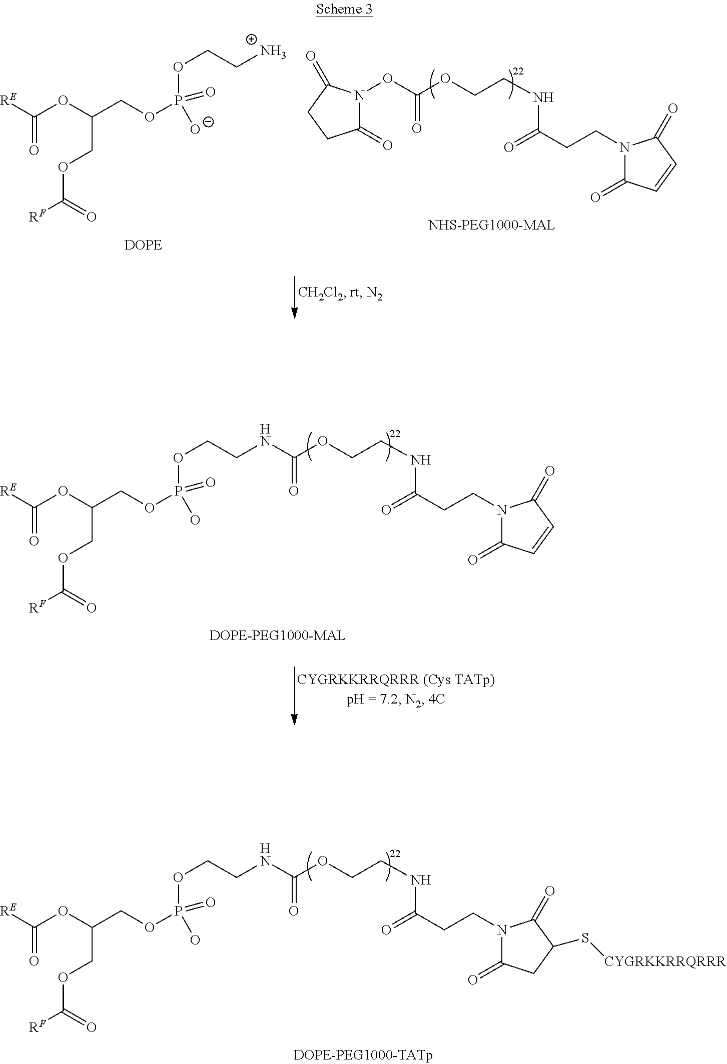

As noted, in some embodiments a micellar composition is provided that includes any of the co-polymers disclosed herein, and further includes an amphiphilic compound, such as any disclosed herein, to form mixed micelles. In some embodiments, the micellar composition has a ratio of co-polymer:amphiphilic compound of about 1:0.001 to about 1:1000. In some embodiments, the micellar composition has a ratio of co-polymer:amphiphilic compound of about 1:0.01 to about 1:100. In some embodiments, the micellar composition has a ratio of co-polymer:amphiphilic compound of about 1:0.1 to about 1:10. In some embodiments, the micellar composition has a ratio of co-polymer:amphiphilic compound of about 1:0.5 to about 1:2. In some embodiments, the micellar composition has a ratio of co-polymer:amphiphilic compound of about 1:0.5. In some embodiments, the micellar composition has a ratio of co-polymer:amphiphilic compound of about 1:0.75. In some embodiments, the micellar composition has a ratio of co-polymer:amphiphilic compound of about 1:1. In some embodiments, the micellar composition has a ratio of co-polymer:amphiphilic compound of about 1:2. In some embodiments, the micellar composition has a ratio of co-polymer:amphiphilic compound of about 1:5. In some embodiments, the micellar composition has a ratio of co-polymer:amphiphilic compound of about 1:10.

### Pharmaceutical Compositions

In another aspect, compositions e.g., “pharmaceutical compositions” are provided including and an effective amount of a co-polymer as described herein. In some embodiments, the composition further includes at least one pharmaceutically acceptable excipient.

Pharmaceutical compositions including the co-polymer or a micelllar composition that includes the co-polymer, as described herein, can be formulated for different routes of administration, including intravenous, intraarterial, pulmonary, rectal, nasal, vaginal, lingual, intramuscular, intraperitoneal, intracutaneous, transdermal, intracranial, subcutaneous and oral routes. Other dosage forms include tablets, capsules, pills, powders, aerosols, suppositories, parenterals, and oral liquids, including suspensions, solutions and emulsions. All dosage forms may be prepared using methods that are standard in the art (see e.g., Remington's Pharmaceutical Sciences, 16^{th} ed., A. Oslo editor, Easton Pa. 1980).

In some embodiments, the co-polymer or a micelllar composition that includes the co-polymer is formulated in conjunction with appropriate salts and buffers to render delivery of the compositions in a stable manner to allow for uptake by target cells. Buffers also are employed when the co-polymer or micelles are introduced into a patient. In some embodiments, an aqueous composition is used, including an effective amount of the co-polymer or a micelllar composition that includes the co-polymer, dispersed in a pharmaceutically acceptable carrier or excipient an aqueous medium. As used herein, “pharmaceutically acceptable carrier” includes any and all solvents, dispersion media, coatings, antibacterial and antifungal agents, isotonic and absorption delaying agents and the like. Sterile phosphate-buffered saline is one example of a pharmaceutically suitable excipient. Other suitable carriers and excipients are well-known to those in the art, see, for example, Ansel et al., PHARMACEUTICAL DOSAGE FORMS AND DRUG DELIVERY SYSTEMS, 5th Edition (Lea & Febiger 1990), and Gennaro (ed.), REMINGTON'S PHARMACEUTICAL SCIENCES, 18th Edition (Mack Publishing Company 1990), and revised editions thereof.

The co-polymer or a micelllar composition that includes the co-polymer may be administered parenterally or intraperitoneally or intratumorally. Solutions of the active compounds as free base or pharmacologically acceptable salts are prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

### Methods of Use

Also provided is an effective method of using the copolymers or micelllar composition that include the co-polymer for delivering small molecule drugs to the interior of target cells (e.g., cancer cells). Thus, in some embodiments, methods of therapy are provided that include or require delivery of small molecule drugs into a cell. In some embodiments, the small molecule drug is a chemotherapeutic drug.

For example, in one non-limiting embodiment, the co-polymer will be formulated in 1 ml of aqueous solution having up to about 50 mg of micelle-forming or mixed micelle forming components (e.g., co-polymers).

Without being bound by theory, the generally hydrophobic drug is expected to solubilize with the hydrophobic lipid portion of the micelle or mixed micelle.

In another aspect, a method for treating cancer in a subject is provided, where the method includes administering to the subject an effective amount of a composition including any of the co-polymers or micelllar compositions described herein.

In another aspect, a method for delivering an antineoplastic drug into one or more cells of a subject is provided, where the method includes administering to the subject an effective amount of a composition including any of the co-polymers or micelllar compositions described herein.

In another aspect, a method is provided for treating cancer in a subject including administering to the subject an effective amount of any of the co-polymers or micellar compositions described herein.

The co-polymers or micelles described herein can be formulated for intravenous administration via, for example, bolus injection or continuous infusion. In some embodiments, the targeted delivery complex is infused over a period of less than about 4 hours; in some embodiments, the infusion is over a period of less than about 3 hours. For example, the first 25-50 mg could be infused within 30 minutes, even 15 min, and the remainder infused over the next 2-3 hrs. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Additionally or alternatively, the active ingredient can be in powder form for constitution with a suitable vehicle, e.g., sterile pyrogen-free water, before use.

The co-polymers or micelles described herein may also be administered to a subject subcutaneously or by other parenteral routes. Moreover, the administration may be by continuous infusion or by single or multiple boluses. In some embodiments, co-polymers or micelles is infused over a period of less than about 4 hours, or over a period of less than about 3 hours.

More generally, the dosage of an administered co-polymer or micelle for humans will vary depending upon such factors as the patient's age, weight, height, sex, general medical condition and previous medical history.

Cell proliferative disorders, or cancers, contemplated to be treatable with the methods include human sarcomas and carcinomas, including, but not limited to, fibrosarcoma, myxosarcoma, liposarcoma, chondrosarcoma, osteogenic sarcoma, chordoma, angiosarcoma, endotheliosarcoma, lymphangiosarcoma, Ewing's tumor, lymphangioendotheliosarcoma, synovioma, mesothelioma, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease.

In some embodiments, the method is used to inhibit growth, progression, and/or metastasis of cancers, in particular those listed above.

The present technology thus generally described will be understood more readily by reference to the following examples which are provided by way of illustration and are not intended to be limiting of the present technology.

### Examples

Materials. Maleimide-polyethylene glycol 1000-N-hydroxysuccinimide ester (MAL-PEG1000-NHS) was purchased from Quanta BioDesign, Ltd. (Powell, Ohio). Polyethylene glycol 2000-N-hydroxysuccinimide ester (PEG2000-NHS) was purchased from Laysan Bio, Inc. (Arab, Ala.). 1,2-distearoyl-sn-glycero-3-phosphoethanolamine-N-[methoxy(polyethylene glycol)-1000] (ammonium salt) (PEG1000-PE), 1,2-dioleoyl-sn-glycero-3-phosphoethanolamine (DOPE), 1,2-dioleoylsn-glycero-3-phosphoethanolamine-N-(lissamine rhodamine B sulfonyl) (ammonium salt) (rhodamine-PE), and 1,2-dipalmitoyl-sn-glycero-3-phosphoethanolamine-N-(7-nitro-2-1,3-benzoxadiazol-4-yl) (ammonium salt) (NBD-PE) were purchased from Avanti Polar Lipids, Inc. (Alabaster, Ala.). Cysteine-modified TAT (Cys-TATp) and MMP2-cleavable (GPLGIAGQ) and uncleavable (GGGPALIQ) octapeptides were synthesized by the Tufts University Core Facility (Boston, Mass.). The BCA Protein Assay Kit, N-hydroxysuccinimide (NHS), triethylamine (TEA), chloroform, dichloromethane (DCM) and methanol were purchased from Thermo Fisher Scientific (Rockford, Ill.). 4-Dimethylaminopyridine (DMAP), N,N′-Dicyclohexylcarbodiimide (DCC), Ninhydrin Spray reagent, Molybdenum Blue Spray reagent, HISTOPAQUE®-1083, Triton® X100 (for electrophoresis), sodium salicylate and propidium iodide were purchased from Sigma-Aldrich Chemicals (St. Louis, Mo.). Human active MMP2 protein (MW 66,000 Da) and TLC plate (silica gel 60 F254) were from EMD Biosciences (La Jolla, Calif.). Dialysis tubing (MWCO 2,000 Da) was purchased from Spectrum Laboratories, Inc. (Houston, Tex.). Dulbecco's modified Eagle's medium (DMEM), penicillin streptomycin solution (PS) (100×), Simply Blue™ Safe Stain (Coomassie® G-250), Hoechst 33342, Annexin V Alexa Fluor® 488 Conjugate, normal mouse sera and trypsin-EDTA were from Invitrogen Corporation (Carlsbad, Calif.). FBS was purchased from Atlanta Biologicals (Lawrenceville, Ga.). SDS-PAGE precast gel (4-20%) was purchased from Expedeon Ltd. (San Diego Calif.). Ready Gel Zymogram Gel (10% polyacrylamide gel with gelatin), Zymogram Renaturation Buffer, Zymogram Development Buffer and the broad range molecular weight standards were purchased form Bio-Rad (Hercules, Calif.). Amicon® Ultra-0.5 centrifugal filter device (100K MWCO) and Fluorescent FragEL™ DNA Fragmentation Detection Kit were purchased from EMD Millipore Corporation (Billerica, Mass.). Cytotox 96 Non-Radioactive Cytotoxicity kit was purchased from Promega Corporation (Madison, Wis.). Collagenase D was from Roche Diagnostics (Indianapolis, Ind.). ALT and AST assay kits were from Biomedical Research Service (Buffalo, N.Y.). ECV304 cell lysate and mouse monoclonal anti-β-Tubulin antibody were purchased from Santa Cruz Biotech (Dallas, Tex.). Human MMP2 ELISA Kit was purchased from Boster Immunoleader (Fremont, Calif.). The mouse plasma was purchased from Bioreclamation (Hicksville, N.Y.). The donkey anti-mouse IgG FITC conjugated antibody was purchased from Jackson ImmunoResearch Laboratories (West Grove, Pa.). The BCA Protein Assay Kit was purchased from Pierce.

### Example 1

### Synthesis, Purification and Characterization of PEG2000-Peptide-PTX

The MMP2-cleavable octapeptide and PEG2000-NHS (1.2:1, molar ratio) were first mixed and stirred in carbonate buffer (pH 8.5) under nitrogen protection at 4° C. overnight. The unreacted peptide was removed by dialysis (MWCO 2,000 Da) against distilled water and checked by RP-HPLC.

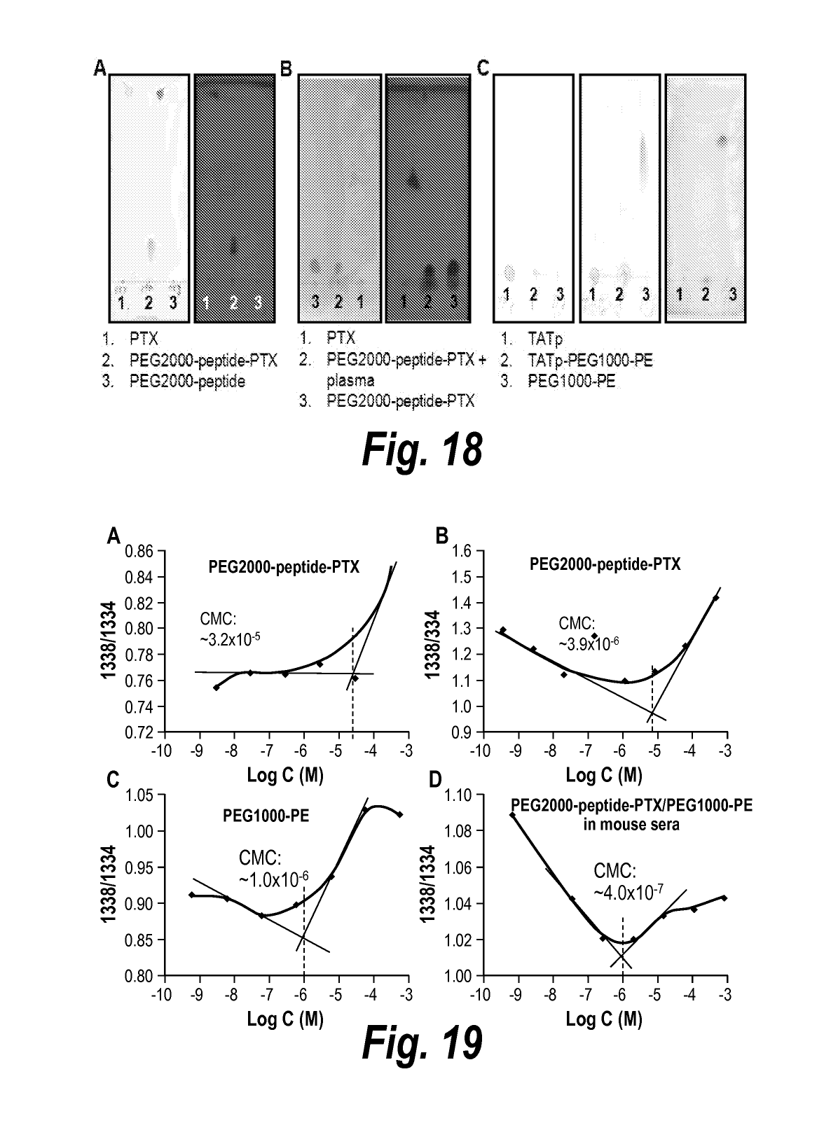

PEG2000-peptide was then activated with a 20-fold molar excess of DCC/DMAP in DCM. Then, a 2-fold molar excess of paclitaxel was added and the reaction was carried out under nitrogen in the dark at room temperature overnight. The reaction was monitored using analytical TLC (chloroform/methanol, 6:4, v/v) and visualized by UV at 254 nm and Dragendorff's reagent (a self-preparation using a U.S. Pharmacopeia protocol) staining. In thin layer chromatography (TLC) (FIG. 18A), a new spot was visualized by both UV and Dragendorff's reagent staining with a significantly lowered retardation factor (R_{f}) value than that of PTX due to the increased hydrophilicity. The product was purified by preparative TLC (same conditions as analytical TLC) and characterized by ¹H-nuclear magnetic resonance (NMR) spectroscopy on a Varian 400 MHz spectroscope with CDCl₃ and D₂O as solvents. To prepare the uncleavable PTX conjugate, the scramble peptide (GGGPALIQ) was used.

The ¹H-NMR spectra of PEG2000-peptide-PTX was obtained in both CDCl₃ and D₂O. The characteristic peaks of the PTX conjugate were clearly displayed when CDCl₃ was used as solvent. PTX is characterized with aromatic (7.35-7.55 ppm), NH (7.0 ppm), acetyl (2.17 and 2.35 ppm), and methyl protons (1.6-1.7 and 1.25 ppm). PEG is characterized by —CH₂CH₂O— protons (3.65 ppm). The peaks of CH₃—, CH₂— and CH— protons in the octapeptide can be found at 1.55-1.75, 1.25 and 0.8 ppm. However, most of PTX peaks disappeared when D₂O was used as solvent. The disappearance of PTX peaks in water could be due to the formation of “core-shell” structure in which the hydrophobic PTX is entrapped in its “core” and isolated by the hydrophilic PEG “shell”, whereas the conjugate would be fully dissolved in chloroform. The integration of the characteristic peaks showed that the molar ratio between PEG (—CH₂CH₂O—) and PTX (aromatic protons) was about 1:1. After reaction, the content of PTX per conjugate was about 24% (w/w) based on its molecular weight.

### Example 2

### Synthesis, Purification and Characterization of TATp-PEG1000-PE

The hetero-bifunctional PEG derivative, NHS-PEG1000-MAL, reacted with DOPE (1:1.4, molar ratio) in DCM. The DOPE-PEG1000-MAL was purified by preparative TLC (same conditions as above). Then, the DOPE-PEG1000-MAL and Cys-TATp (CYGRKKRRQRRR) (1:1.2, molar ratio) were mixed in a pH 7.2 HEPES buffer and the reaction was carried out at 4° C. under nitrogen protection overnight, followed by dialysis (MWCO 2,000 Da) against distilled water to remove unreacted TATp. The reaction and purification processes were monitored by TLC (chloroform/methanol, 8:2, v/v) and visualized using Dragendorff's reagent staining for PEG, Ninhydrin Spray reagent staining for peptides, and Molybdenum Blue Spray reagent staining for phospholipids. (FIG. 18C).

The co-polymers may be characterized for size and uniformity by any suitable analytical techniques. These include, but are not limited to, atomic force microscopy (AFM), electrospray-ionization mass spectroscopy, MALDI-TOF mass spectroscopy, ¹³C nuclear magnetic resonance spectroscopy, high performance liquid chromatography (HPLC) size exclusion chromatography (SEC) (equipped with multi-angle laser light scattering, dual UV and refractive index detectors), capillary electrophoresis and get electrophoresis.

### Example 3

### Characterization of the Micelle Formation

To study physicochemical properties of PEG2000-peptide-PTX, the particle size, morphology, and critical micelle concentration (CMC) were analyzed (FIG. 2 and FIG. 19), as discussed below. The CMC of PEG2000-peptide-PTX was about 3.2×10⁻⁵ M, which is in the range of the CMC of the micelles formed by PEG2000-PE, indicating the formation of a “core-shell” structure. The transmission electron microscopy (TEM) showed that PEG2000-peptide-PTX formed non-spherical particles with a size of 61.3±15 nm. This suggests that the hydrophobic interaction/force among PTX molecules is not strong enough to “hold” them together tightly, resulting in a large and loose “core-shell” structure. In contrast, PEG1000-PE containing a strong hydrophobic lipid moiety formed uniform micelles with a spherical shape, small size (11.9±2.0 nm) and low CMC (1×10⁻⁶ M). Mixing PEG1000-PE with PEG2000-peptide-PTX faciliates the micelle formation as evidenced by a decreased CMC (3.9×10⁻⁶ M), near-spherical shape, and intermediate size (22.5±2.7 nm). The measured CMC of PEG2000-peptide-PTX/PEG1000-PE micelles was similar to the theoretical CMC (1.9×10⁻⁶ M) using the equation: 1/CMC=X₁/CMC₁+X₂/CMC₂, suggesting the formation of a mixed micelle. Compared to the CMC obtained in the serum-free medium, the CMC of PEG2000-peptide-PTX/PEG1000-PE in the presence of serum was even lower (4×10⁻⁷ M), probably due to the high ionic strength of the serum. The increased ionic strength usually decreases the CMC of the amphiphilic polymers, such as lipids and lipid derivatives, which could be well explained by the “binding model” theory. The low CMC of the nanopreparation ensures the in vitro and in vivo stability of these core-shell/micellar structures.

### Example 4

### Determination of Critical Micelle Concentration (CMC)

The CMC was determined by fluorescence spectroscopy using pyrene as a hydrophobic fluorescent probe. Briefly, pyrene chloroform solution was added to the testing tube at the final concentration of 8×10⁻⁵M and dried on a freeze-dryer overnight. Then, the nanopreparation in Hank's Balanced Salt Solution (HBSS) was added to the tubes at the ten-fold serial dilutions (from 1 to 10⁻⁷ mg/mL) and incubated with shaking at room temperature for 24 h before measurement. To study the influence of the serum on the CMC, the nanopreparation was hydrated by HBSS containing 50% mouse serum. The fluorescence intensity was measured on an F-2000 fluorescence spectrometer (Hitachi, Japan) with the excitation wavelengths (λₑₓ) of 338 nm (I₃) and 334 nm (I₁) and an emission wavelength (λₑₘ) of 390 nm. The intensity ratio (1338/1334) was calculated and plotted against the logarithm of the micelle concentration. The CMC value was obtained as the crossover point of the two tangents of the curves.

### Example 5

### Preparation of the MMP2-Sensitive Nanopreparation (i.e., Micelles)

To prepare the MMP2-sensitive nanopreparation (TATp-PEG1000-PE/PEG2000-peptide-PTX), PEG2000-peptide-PTX (50 mol %), PEG1000-PE (40 mol %), and TATp-PEG1000-PE (10 mol %) were dissolved in chloroform and dried on a freeze-dryer overnight, followed by hydration with Hank's Balanced Salt Solution (HBSS) at room temperature. The non-sensitive nanopreparation (TATp-PEG1000-PE/PEG2000-peptide-PTX uncleavable), nanopreparations without TATp modification (PEG1000-PE/PEG2000-peptide-PTX and PEG1000-PE/PEG2000-peptide-PTX uncleavable), and the empty micelle (TATp-PEG1000-PE) were prepared using the same method. The particle size and morphology of these nanopreparations were analyzed by transmission electron microscopy (TEM) (model XR-41B) (Advanced Microscopy Techniques, Danvers, Mass.) using negative staining with 1% phosphotunstic acid (PTA). MMP2-cleavable peptide refers to the (GPLGIAGQ) octapeptide and MMP2-uncleavable peptide refers to the (GGGPALIQ) octapeptide.

### Example 6

### Stability of the MMP2-Sensitive Nanopreparation

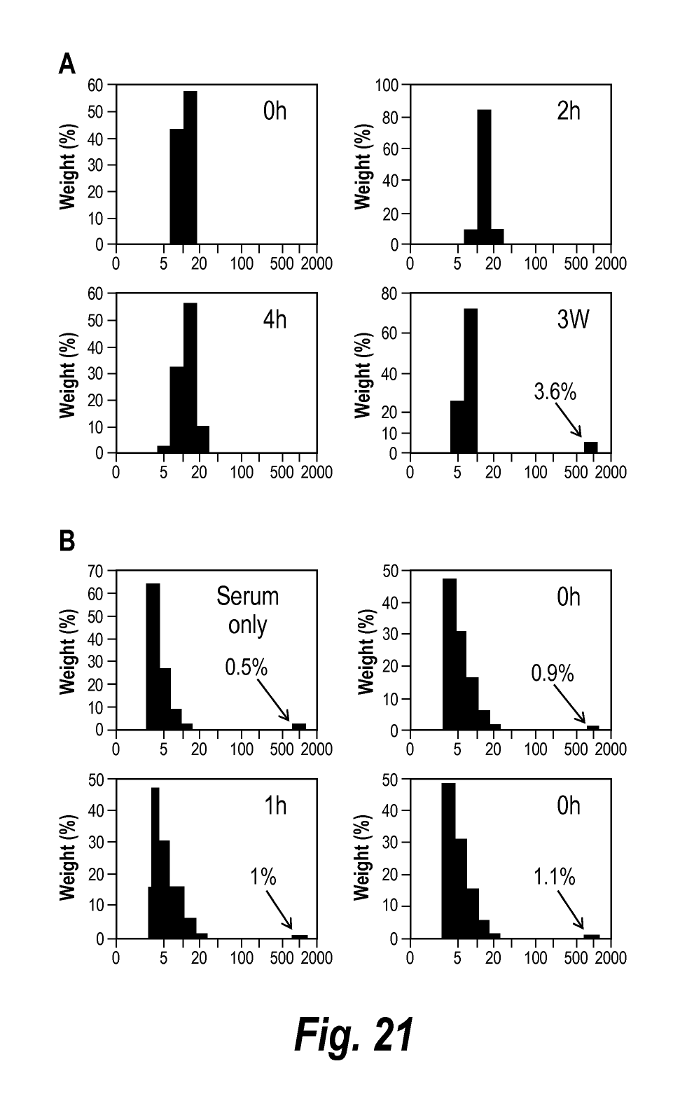

The particle size of the nanopreparations was measured by the dynamic light scattering (DLS) as a measure of stability of the micellar structures. After incubation with Hank's Balanced Salt Solution (HBSS) at 37° C. for 4 h, there was no significant change in the size of the nanopreparation. After long term storage (3 weeks) at 4° C., a slight aggregation (3.6%) was observed (FIG. 21A). These data indicated that the formed micelles were quite stable in the aqueous buffer. In normal mouse sera, the small number of larger aggregates (>500 nm) caused by the interaction of nanopreparations and blood proteins was slightly increased from 0.5% (serum only) to 1.1% after 4 h incubation at 37° C. (FIG. 21B), indicating little in vivo protein adsorption/interaction/opsonization due to the high density of PEG and appropriate PEG length on the surface of the nanopreparation. The MMP2-sensitive nanopreparation with the minimized protein adsorption and small size are more likely to “escape” the capture by immune cells.

### Example 7

### Cleavage of PEG2000-peptide-PTX by MMP2

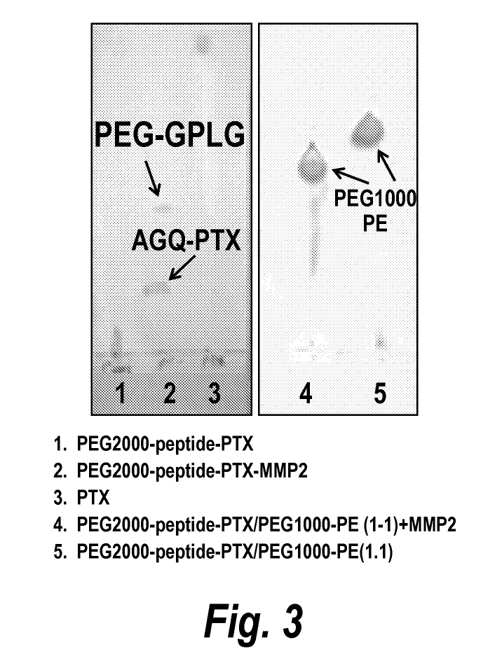

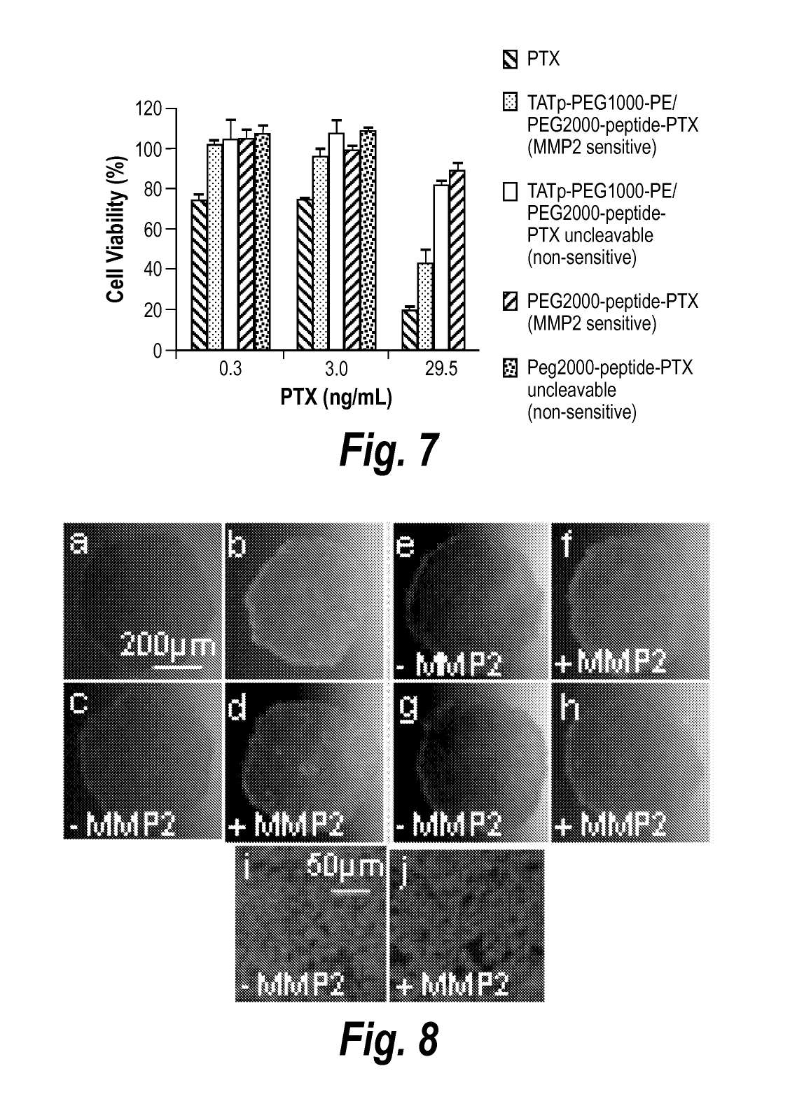

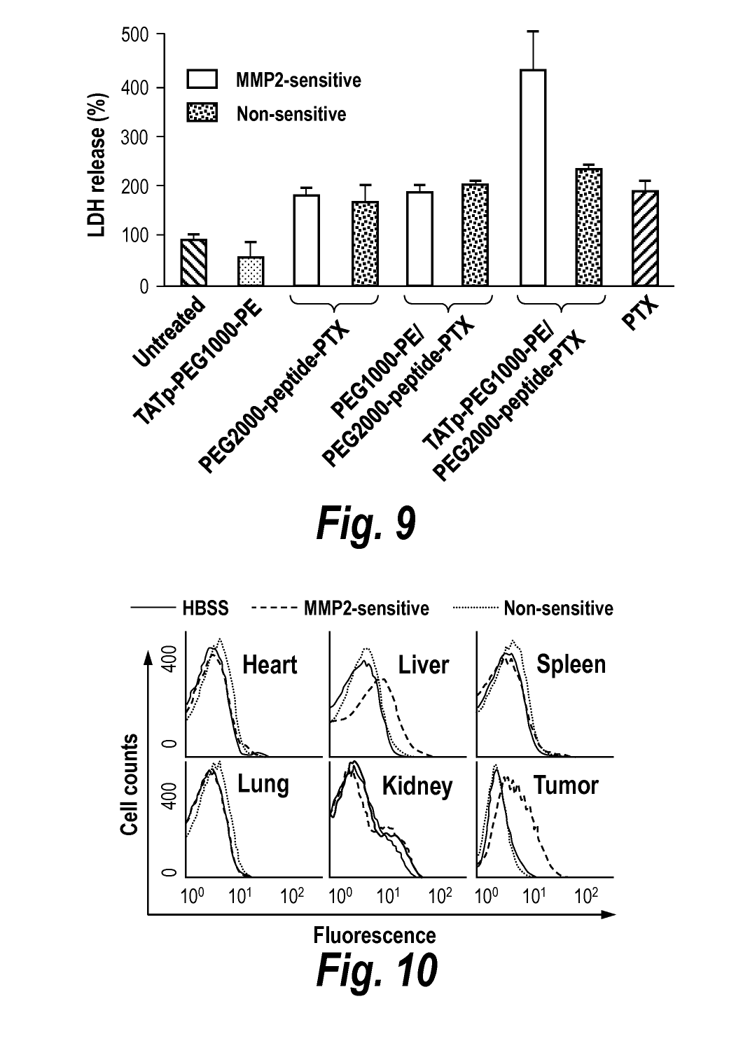

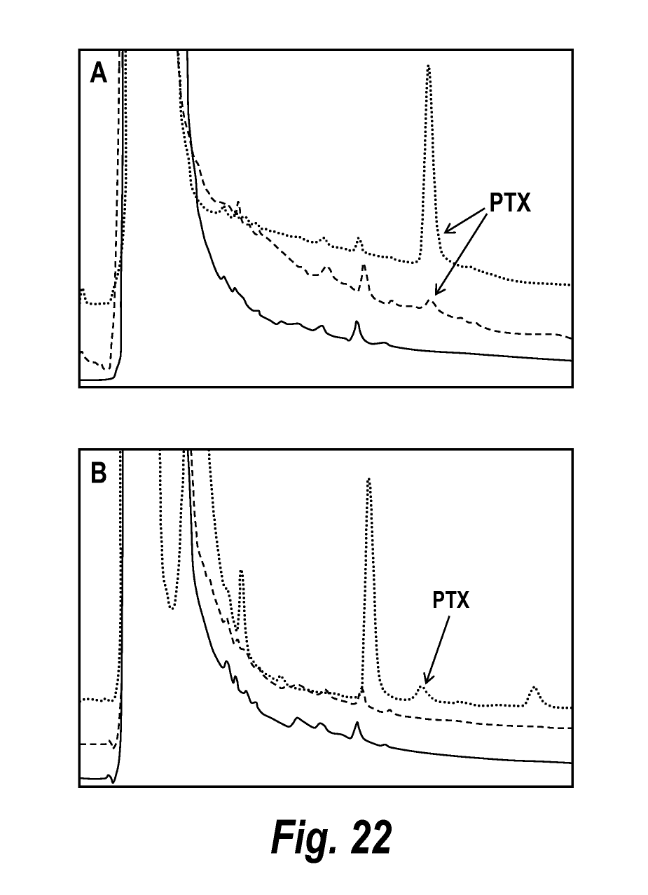

The cleavability of PEG2000-peptide-PTX was determined by enzymatic digestion followed by TLC. After incubation with 5 ng/μL of human MMP2, the spot of the PTX conjugate was disappeared while two new spots were seen in the TLC plate (FIG. 3). This indicated that the MMP2 completely cleaved the peptide linker resulting in two digestion fragments (IAGQ-PTX and PEG-GPLG) Furthermore, incubation of PEG2000-peptide-PTX/PEG1000-PE mixed micelles with MMP2 showed similar results to PEG2000-peptide-PTX alone (FIG. 3), indicating sufficient accessibility of MMP2 to the peptide even in this “compact” micellar structure. In contrast, incubation of PEG2000-peptide-PTX with mouse plasma could not cleave the linker (FIG. 18B). After MMP2-mediated cleavage, the release of IAGQ-PTX from the micelles was analyzed (FIG. 22). After dialysis for 24 h, about 48% of the free PTX was released from the dialysis tube (middle curve, FIG. 22A) while TATp-PEG1000-PE/PEG2000-peptide-PTX micelles with MMP2 pretreatment didn't have any free PTX outside the tube (middle curve, FIG. 22B), similar to the one without MMP2 pretreatment (bottom curve, FIG. 22B). However, the peak of PTX appears after trypsinization of the sample inside the dialysis tube (top curve, FIG. 22B). Trypsin is a potent protease which can digest peptides, and trypsinization removes the peptide residue or at least part of it from IAGQ-PTX. The released PTX showed the same retention time as the “naked” PTX. Although the method is not the most sensitive to quantitate the remaining PTX in the micellar core, the data indicated that, after cleavage, IAGQ-PTX was still incorporated into TATp-PEG 1000-PE/PEG1000-PE micelles due to its high hydrophobicity (FIG. 3). This guaranteed the enhanced cell internalization of IAGQ-PTX, which is mediated by the micelle surface-attached TATp (FIGS. 6 and 8). After internalization, the peptide residue of PTX fragments would be digested/removed by the intracellular enzymes (such as endosomal proteases) and not be the obstacle of the pharmacological activity of the liberated PTX (FIGS. 7 and 9).

### Example 8

### In Vitro Drug Release

To study whether the MMP2 cleavage influences the encapsulation of paclitaxel, the drug release study was performed. Briefly, TATp-PEG1000-PE/PEG2000-peptide-PTX micelles were pre-treated with MMP2 at 37° C. overnight. The samples (0.8 mL) were then dialyzed (MWCO 2,000 Da) against 20 mL of water containing 1M sodium salicylate to maintain the sink condition. The PTX in a methanol/water (1:4, v/v) mixture and TATp-PEG1000-PE/PEG2000-peptide-PTX micelles without MMP2 pretreatment were used as controls. The PTX in the outside and inside media was determined by RP-HPLC. To obtain the naked PTX (without amino acid residues), the samples were trypsinized at 37° C. for 1 h before HPLC.

### Example 9

### Determination of PTX by RP-HPLC

The samples were analyzed by RP-HPLC on a reverse phase C18 column (250 mm×4.6 mm, Alltech) by the LaChrom Elite HPLC system (Hitachi). The chromatograms were collected at 227 nm using acetonitrile/water (45:55, v/v) as the mobile phase at a flow rate of 1.0 mL/min.

### Example 10

### Tubulin Immunostaining

1.2×10⁵ cells were seeded on glass coverslips in a 12-well plate and incubated for 24 h before treatment. Cells were then incubated with 24 nM of PTX formulations at 37° C. overnight. The treated cells were washed with PBS and fixed with 4% paraformaldehyde (PFA) followed by the permeabilization with 0.5% Triton X-100 at room temperature. After washing with PBS, the cells were incubated with a mouse monoclonal anti-β-tubulin antibody at a 1:10 dilution in PBS containing 1% BSA at room temperature for 1 h. Cells were then washed three times with PBS before staining with a donkey anti-mouse IgG FITC conjugated antibody at a 1:100 dilution at room temperature for 1 h. Finally, the cells were washed with PBS and stained with Hoechst 33342 before confocal microscopy.

### Example 11

### Establishment of A549 Tumor Cell Spheroids

A549 multicellular spheroids were formed. Briefly, A549 cells were grown in DMEM supplemented with 50 U/mL penicillin, 50 μg/mL streptomycin and 10% FBS at 37° C. The 96-well plates were coated with 1.5% agarose in DMEM to prevent cell adhesion and seeded with 1×10⁴ cells per well. The plates were then centrifuged at 1,500 rpm for 15 min and the multicellular aggregates were maintained at 37° C. for spheroid formation. The spheroid was identified by its size and shape. The 6-day spheroids with a diameter of 700-900 μm were used as the in vitro tumor model.

### Example 12

### TATp Competition Study

A549 cells were seeded in 24-well plates at 1.6×10⁵ cells/well in 300 μL/well of complete growth media. After 24 h, the free TATp (0.35, 3.5 or 35 μM) was added into the cell media. Then, 10 μL of the rhodamine-PE-labeled MMP2-sensitive nanopreparation (1 mg/mL) was immediately added and incubated for 2 h. The media was removed and the cells were washed with serum-free media three times. Cells were then analyzed by FACS.

### Example 13

### Apoptosis Analysis

The apoptosis of A549 cells was determined by FACS using Annexin V/Propidium Iodide double staining after treatment with PTX or its conjugate at 29.5 ng/mL for 72 h according to the manufacturer's instruction. Briefly, treated cells were trypsinized and collected by centrifugation at 1,500 rpm for 5 min. The cells were washed and re-suspended with 100 μL PBS. The cells were first stained with Annexin V (25 μg/mL) for 15 min on ice, and then incubated with propidium iodide (50 μg/mL) for 5 min before FACS analysis.

### Example 14

### MMP2-Triggered Tumor Cell-Specific Cytotoxicity of PEG2000-Peptide-PTX

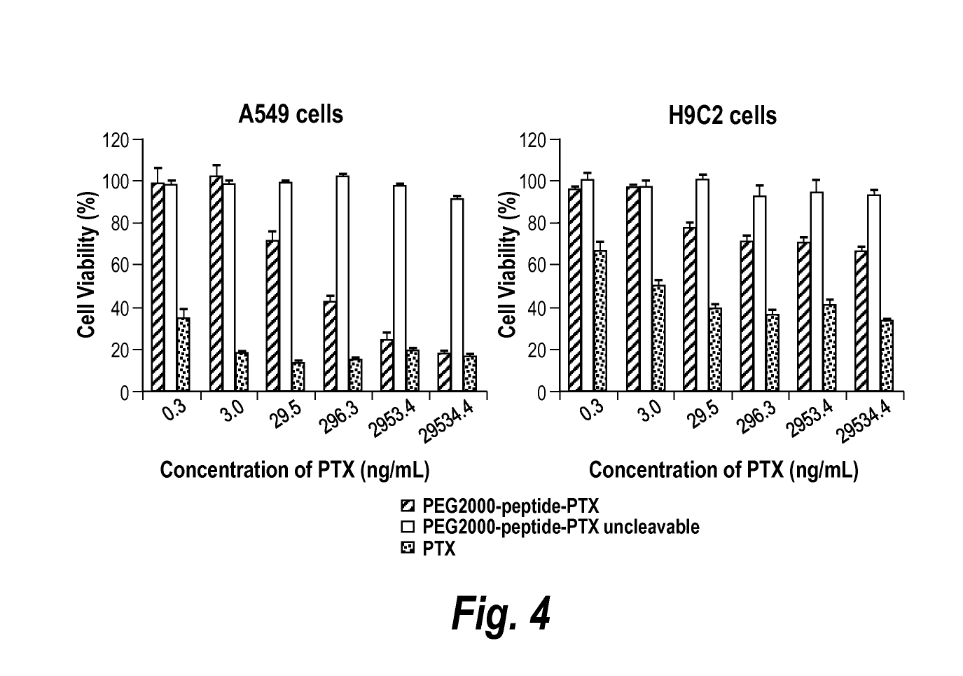

PEG2000-peptide-PTX and its uncleavable counterpart were tested in A549 tumor cells and H9C2 normal cardiomyocytes (FIG. 4). The decreased cytotoxicity of PTX was observed in both cell lines after PEGylation. In tumor cells, PTX and PEG2000-peptide-PTX showed comparable strong toxicity (around 20% cell viability) at high doses, while PEG2000-peptide-PTX was much safer in normal cells. Higher cytotoxicity of PTX in A549 cells than H9C2 cells is understandable since tumor cells have higher proliferation rates than normal cells, resulting in the different response to the same treatment.

The cytotoxicity of PTX and its conjugate was dose-dependent. This indicated that the released IAGQ-PTX was still cytotoxic after the MMP2-mediated cleavage. Esterification at either C-2′ or C-7′ did not significantly influence PTX's activity. The cytotoxicity of PEG2000-peptide-PTX was lower than that of free PTX, since PEG inhibits the cellular uptake of the conjugate (FIG. 6), and only released PTX fragments can be efficiently taken up by cells.

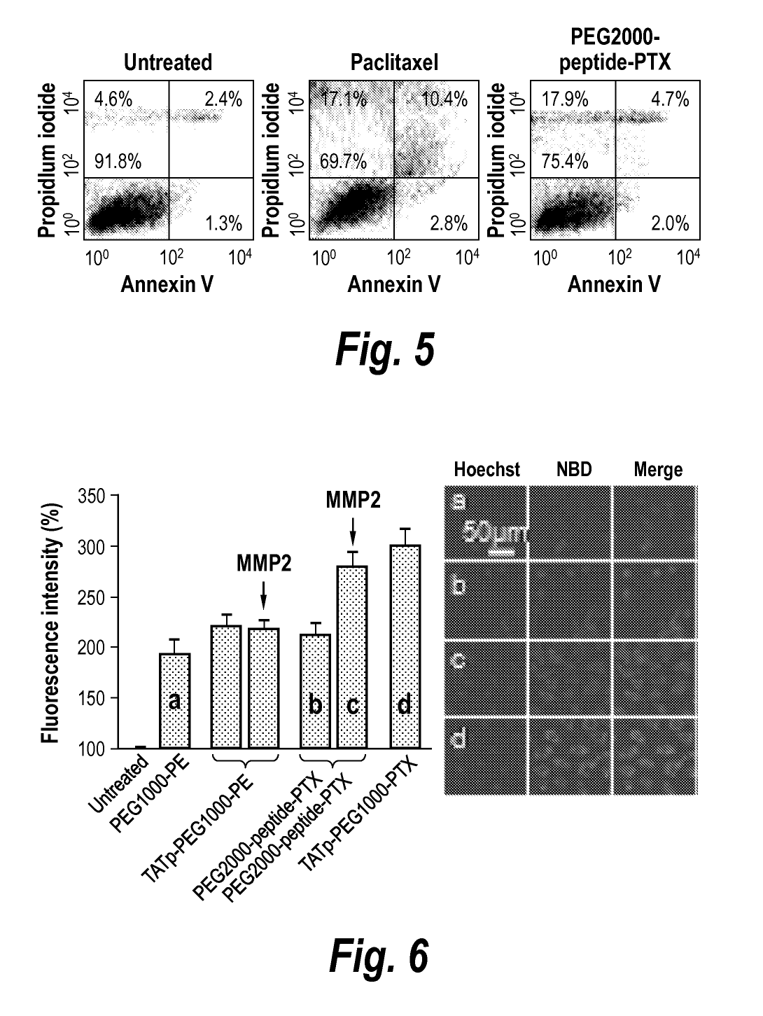

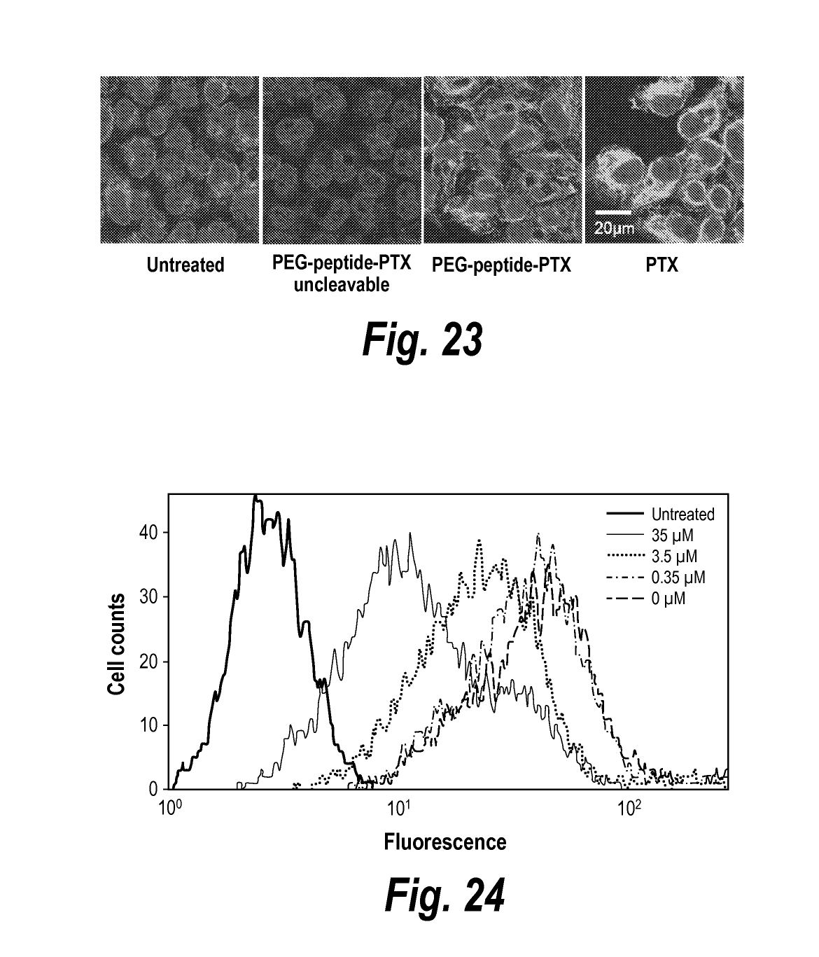

The apoptosis-inducing ability of PEG2000-peptide-PTX was analyzed by fluorescence-activated cell sorting (FACS) (FIG. 5). The percentage of viable cells of PTX (69.7%) and its conjugate (75.4%) treated groups was much lower than that of untreated cells (>90%). The similar percentage of early apoptotic cells (Annexin positive only) was detected in PTX (2.8%) and PEG2000-peptide-PTX (2.0%) groups. As expected, the percentage of late apoptotic cells (both Annexin and PI positive) in PEG2000-peptide-PTX was lower than that of PTX (4.7% vs. 10.4%), but it is still much higher than that of untreated cells (2.3%). Both treatments significantly increased the number of necrotic/dead cells (PI positive only) with 17.1% for PTX and 17.9% for its conjugate compared to only 4.6% in untreated cells. To further clarify the mechanism of the PTX conjugates, the treated cells were stained by the monoclonal anti-β-tubulin antibody. PEG2000-peptide-PTX induced a significant tubulin polymerization, as evidenced by the visualized green fluorescent filaments around cell nuclei, compared to the uncleavable PTX conjugate and untreated cells. However, the fluorescence intensity was somewhat lower than that of free PTX-treated cells (FIG. 23). In our design, this decreased activity of PEG2000-peptide-PTX caused by PEGylation was used to minimize the non-specific cytotoxicity of the PTX conjugate to normal cells (FIG. 4). To exert effective anticancer effects, the decreased activity can be easily compensated by a higher dose (FIG. 4), appropriate nanocarriers (FIG. 7), or a longer treatment time (FIG. 9). By contrast, the uncleavable PTX conjugate did not show cytotoxicity with all doses in either cell line (FIG. 4).

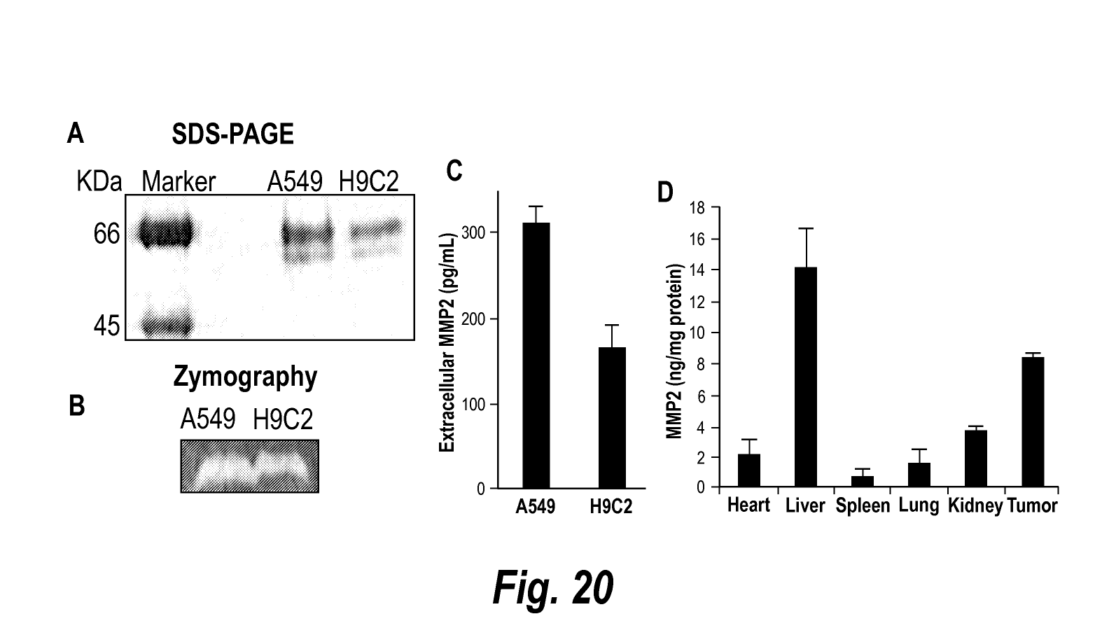

These data are consistent with the extracellular MMP2 levels (FIG. 20 A-C). Both cell types secreted proteins with gelatinase activity and a molecular weight close to active MMP2 (66.5 KDa, EMD Biosciences). The MMP2 level in A549 cell media was much higher than that from H9C2 cells, and efficiently cleaved the peptide linker, allowing PTX liberation from its nontoxic prodrug. The normal cardiomyocyte was selected as the control because of its low extracellular MMP2 level. The cytotoxicity of PEG2000-peptide-PTX in normal cells is probably due to the basal MMP2, which is required to maintain a cell's normal activity. An induction/activation of MMP2 in normal cells by the toxic chemotherapeutics like PTX and doxorubicin is possible. However, compared to tumoral MMP2, its influence on the PTX conjugate is limited. These data suggested that PEG2000-peptide-PTX has MMP2-triggered tumor cell-specific cytotoxicity.

### Example 15

### Cellular Uptake and Cytotoxicity of the MMP2-Sensitive Nanopreparation

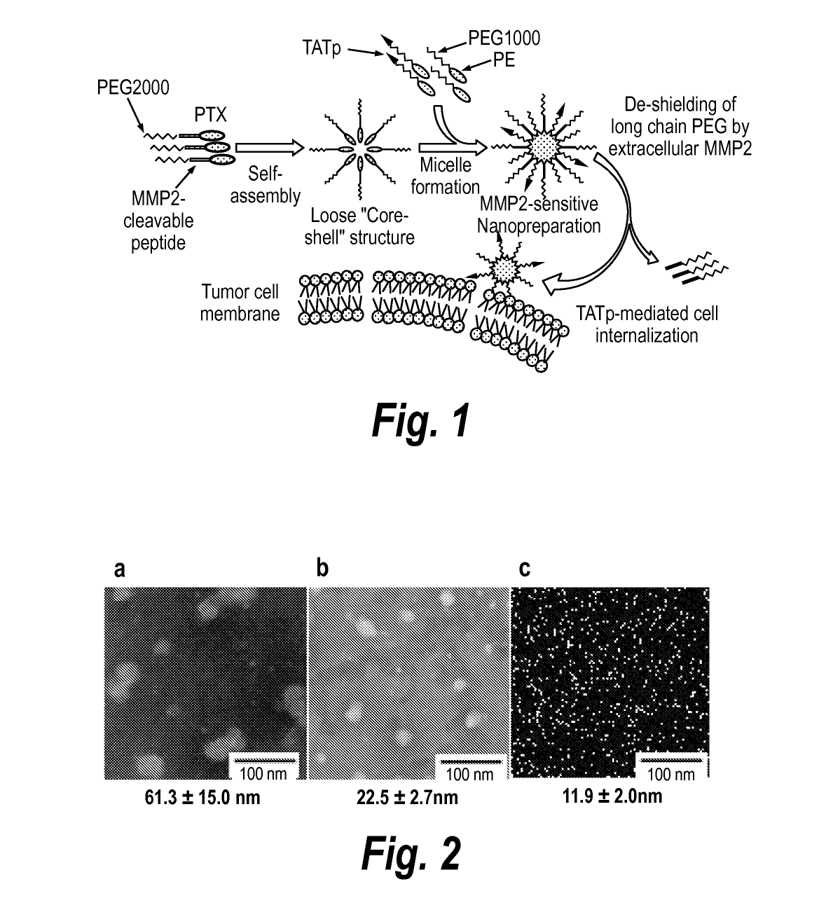

The self-assembled MMP2-sensitive nanopreparation composed of PEG2000-peptide-PTX, PEG1000-PE and TATp-PEG1000-PE (FIG. 1) had a relatively high drug loading (15% w/w) and stable structure, which is superior to most conventional PTX polymeric micelles with low drug loading [usually, less than 5% w/w] and a higher risk of drug leakage.

The cellular uptake of NBD-PE-labeled nanopreparations was evaluated by FACS and confocal microscopy (FIG. 6). PEG2000 in the MMP2-sensitive nanopreparation prevented the TATp-mediated cell internalization (b), which was restored to the level (c) similar to that obtained with TATp-PEG1000-PE micelles (d) after MMP2-induced cleavage (FIG. 6). Since TATp's role in nanocarriers has been systematically evaluated in our previous studies and this work might be considered as one of its applications, the competition effect of the free TATp was examined to confirm the function of TATp in this nanopreparation. The data showed that adding free TATp to cell media decreased the TATp-mediated cellular uptake of rhodamine-PE-labeled nanopreparations. The competition effect was dose-dependent (FIG. 24). As a result of the enhanced cellular uptake, the MMP2-sensitive nanopreparation killed more tumor cells (44% cell viability, at 29.5 ng/mL) compared to its non-sensitive counterpart, PTX conjugate and uncleavable conjugate (FIG. 7).

However, the two-dimensional cell culture cannot fully represent in vivo tumors since they are different in terms of cellular heterogeneity, nutrient and oxygen gradients, cell-cell interactions, matrix deposition and gene expression profiles, resulting in different drug responses and poor in vitro-in vivo correlation. To better mimic the real tumor conditions in vitro, A549 multicellular spheroids were established to study the penetration and cell internalization of rhodamine-PE-labeled nanopreparations (FIG. 8). The presence of the long-chain PEG in the nanopreparation lowered its cell association. However, MMP2 pretreatment significantly increased nanopreparations' penetration of spheroids (d, f and h vs. c, e and g). Strong red fluorescence around cell nuclei was clearly shown upon pretreatment of TATp-PEG1000-PE/PEG2000-peptide-PTX with MMP2 (j vs. i), indicating that MMP2-triggered PEG de-shielding allows the exposure of the previously hidden TATp and enhanced cell internalization.

The cytotoxicity of the nanopreparations was also evaluated using this in vitro model (FIG. 9). After 3 treatments at 29.5 ng/mL, all PTX formulations except TATp-PEG 1000-PE/PEG2000-peptide-PTX showed similar cytotoxicity with about 2-fold increase of the lactate dehydrogenase (LDH) release compared to untreated spheroids, while the empty nanocarrier (TATp-PEG1000-PE) showed no cytotoxicity. The limited cytotoxicity of the non-sensitive nanopreparations was probably a result of the non-specific cell internalization and cumulative effect of the treatments, which would not be reproduced in the in vivo dynamic conditions. It was also notable that free PTX didn't cause the highest cytotoxicity, probably due to its poor penetration of the spheroids. By contrast, TATp-PEG 1000-PE/PEG2000-peptide-PTX showed the highest cytotoxicity with a more than 4-fold LDH release.

### Example 16

### Determination of MMP2 Levels in Cell Cultures and Tissues

The human non-small-cell lung cancer cells (A549 cells) and normal rat cardiomyocytes (H9C2 cells) were seeded in a 6-well plate at 1×10⁵ cell/well and maintained in complete growth media for 3 days. Then the cell media was collected and concentrated using an Amicon® Ultra-0.5 centrifugal filter device (30K MWCO) at 10,000 rpm for 20 min. The concentrated media was loaded and run on a 4-20% SDS-PAGE gel followed by Simply Blue™ Safe Stain staining. The broad range molecular weight standards were used as molecular weight markers. The gelatinase activity of the secreted proteins was determined by gelatin zymography using a pre-cast 10% zymogram gel followed by Simply Blue™ Safe Stain staining ECV304 cell lysate was used as positive control for MMP2. For quantitative detection of MMP2, an MMP2 ELISA assay (sensitivity<10 pg/mL) was performed to detect the MMP2 concentration in the original cell media (without concentration process) according to the manufacturer's instruction.

To determine the MMP2 levels in tissues, the tumor-bearing mice were sacrificed and the tumors and major organs were collected. The tissues were then homogenized in PBS containing 0.5% Triton® X100 by a TissueRuptor (QIAGEN) on ice. The homogenates were centrifuged at 2250 rpm for 20 min, and then analyzed by the MMP2 ELISA assay. The concentration of the total protein was measured with a BCA Protein Assay Kit.

### Example 17

### Establishment of the NSCLC Xenograft Mouse Model

Female nude mice (NU/NU, 4-6 weeks old) were purchased form Charles River laboratories (Wilmington, Mass.). All animal procedures were performed according to an animal care protocol approved by Northeastern University Institutional Animal Care and Use Committee. Mice were housed in groups of 5 at 19 to 23° C. with a 12 h light-dark cycle and allowed free access to food and water.

Approximately 2×10⁶ A549 cells suspended in 50 μl HBSS were mixed with the phenol-red free high concentration Matrigel™ (1:1, v/v) and inoculated in nude mice by subcutaneous injection over their right flanks. The tumor was monitored for length (1) and width (w) by caliper and calculated by the equation V=lw²/2.

### Example 18

### In Vivo Tumor Targeting and Antitumor Efficacy

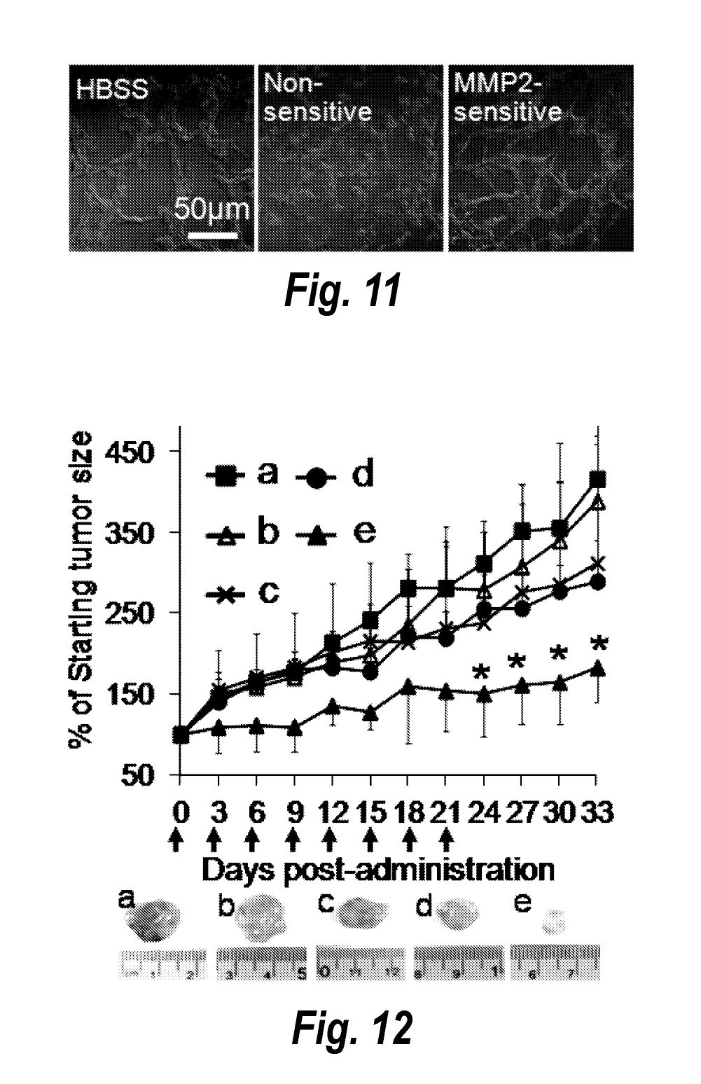

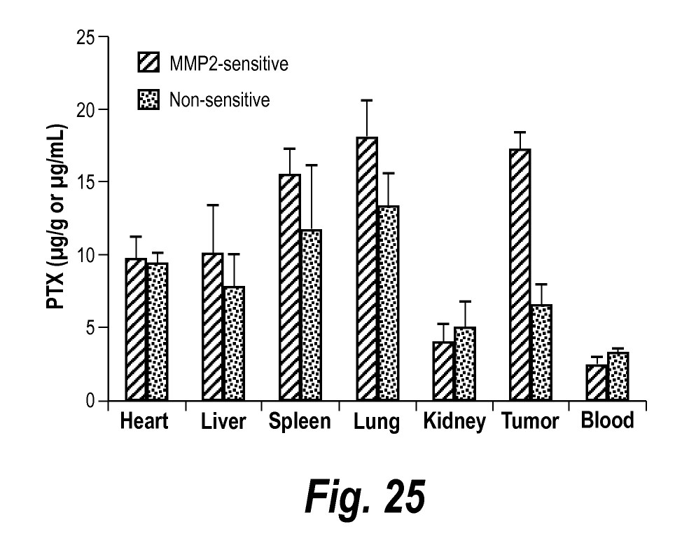

The tumor targetability and antitumor efficacy of the MMP2-sensitive nanopreparation were evaluated in a NSCLC xenograft mouse model. The in vivo cell internalization of rhodamine-PE-labeled nanopreparations was analyzed by flow cytometry after cell dissociation at 2 h after intravenous injection (FIG. 10). No significant fluorescence in heart, spleen, lung and kidney cells was observed after the administration, indicating negligible accumulation of the nanopreparations there. In contrast, the cellular uptake in the liver and tumor was significantly higher, since these tissues contain a large amount of the MMP2 (FIG. 20D). The high MMP2 level might be related to the high cell internalization of the nanopreparation in the liver, while the high tumor accumulation of the nanopreparation was the result of the combination effect of the EPR effect and the up-regulated MMP2 in the tumor. This was confirmed by the enhanced red fluorescence around cell nuclei (blue) in confocal micrographs (FIG. 11). Furthermore, to see the PTX tissue distribution, the PTX concentration in the tumor, organs and blood was measured by RP-HPLC (FIG. 25). No significant difference in the PTX concentration was observed in the major organs and blood between the MMP2-sensitive nanopreparation and non-sensitive one. In contrast, the MMP2-sensitive nanopreparation resulted in a more than 2.5-fold higher PTX concentration in the tumor tissue compared to its non-sensitive counterpart, which is consistent with the in vivo cellular uptake data (FIGS. 10 and 11). The liver didn't show the highest drug accumulation. Instead, the lung and spleen showed the high drug accumulation similar to the tumor. The difference between the two methods is understandable. The tissue accumulation of PTX (HPLC data) showed the overall PTX concentration including both intracellular and extracellular drugs while the in vivo cell internalization data showed only the intracellular nanoparticles/drug. Since the TATp was expected to mediate the enhanced cellular uptake after the MMP2-mediated cleavage, the real in vivo cellular uptake data might be more informative. The tissue accumulation data cannot differentiate the intracellular nanoparticles/drug from the extracellular ones, therefore, might be not enough to fully describe the MMP2-sensitive nanopreparation's in vivo behavior. Although the data obtained from the two methods had different meanings, they were actually consistent and delivered the same information that the MMP2-sensitive nanopreparation had the excellent tumor targetability. A small size and PEG “corona” minimized the distribution/cell internalization of nanoparticles in non-target tissues, while the combined use of the MMP2-sensitive moiety, a cell-penetrating enhancer in the nanoparticles and the tumoral EPR effect enhanced their tumor cell-selective internalization.

Like with most chemotherapeutics, internalization of the nanopreparation in the tumor by non-cancer cells (including immune cells, such as myeloid cells) may occur. However, the population of these cells is much lower than that of tumor cells, and myeloid cells themselves also contribute to the tumor invasion in the aggressive tumors, which makes the MMP2-sensitive nanopreparation efficacious to inhibit the tumor growth. As a secreted soluble protein, the proteolytically active MMP2 is not only residing in the extracellular matrix and circulating in the blood but also attached on the surface of invasive cells, such as cancer cells, by interaction with integrin αvβ3. The cancer cell-surface integrin receptor regulates both cell migration and matrix degradation, facilitating cancer cell's invasion. In addition to the soluble form of MMP2 in the tumor's extracellular matrix, the cell surface-bound MMP2 may also contribute to the MMP2-sensitive tumor-targeted drug delivery.

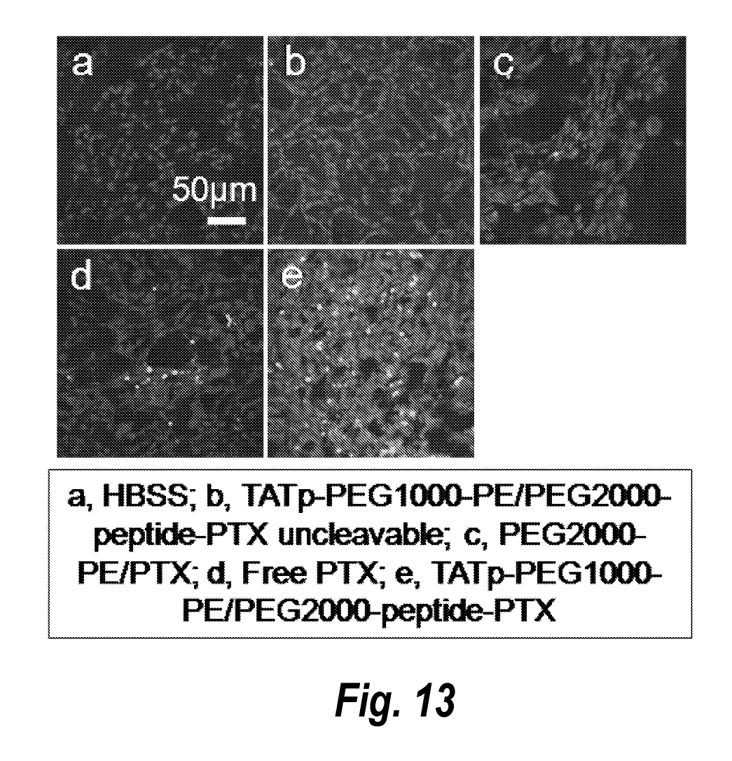

To test the therapeutic activity of the MMP2-sensitive nanopreparation, tumor-bearing mice were injected with PTX formulations twice a week for 4 weeks at the dose of 5 mg/Kg PTX. Our in vitro data clearly showed that PEG1000-PE/PEG2000-peptide-PTX micelles and the formulations without TATp modification or with the blocked TATp could not make improvement in terms of the cellular uptake and antitumor effects, and only the exposed TATp could efficiently enhance the internalization of the nanocarrier by target cells (FIGS. 6, 7, 8 and 9). This conclusion has been repeatedly proved in different nanocarriers including the similar PEG-PE micelles by our and other groups. Therefore, the only absolutely required groups were tested and compared in vivo to decrease the number of used animals. The formulation with the blocked TATp (non-sensitive nanopreparation) and the formulation with the exposed TATp (MMP2-sensitive nanopreparation) were compared to show the difference between the hidden TATp and the exposed one. Besides, the micelle group PEG2000-PE/PTX, which has no TATp modification, could be considered as another negative control for the TATp modification. The tumor growth of the MMP2-sensitive nanopreparation group (e) was significantly inhibited compared to HBSS (a), the non-sensitive nanopreparation (b), PTX conventional micelles (c), and free PTX (d) (FIG. 12). It was also notable that the tumor growth inhibition was well correlated with significant apoptosis seen in tumor tissues (green fluorescent dots in the TUNEL assay) (FIG. 13).

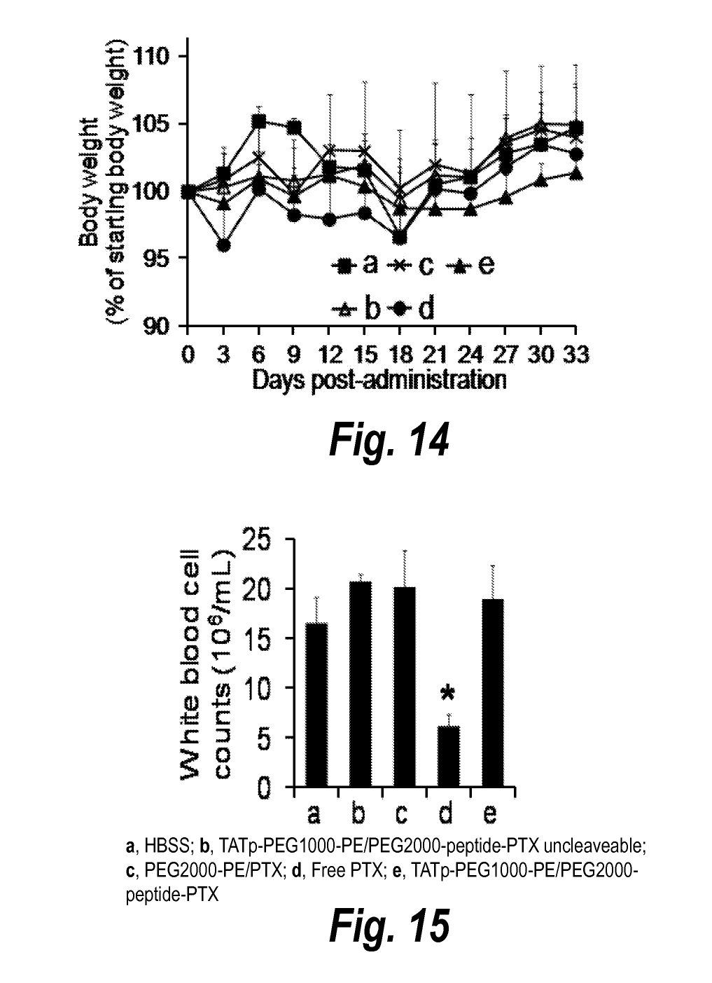

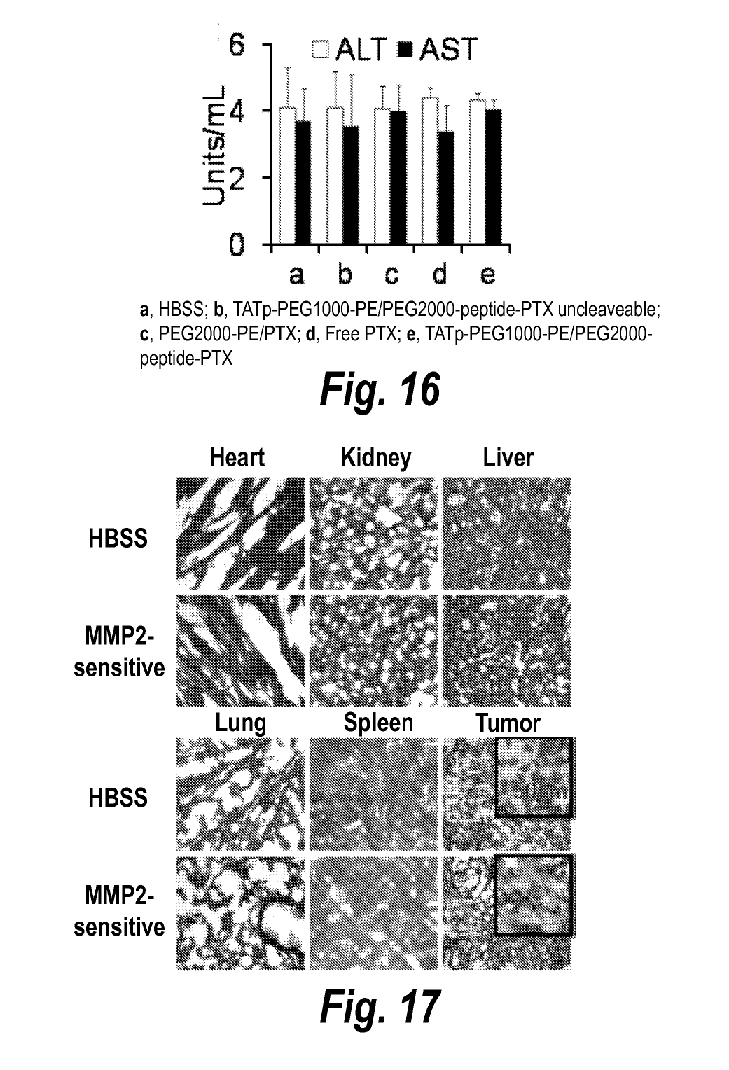

No significant changes were observed after treatment of mice with the MMP2-sensitive nanopreparation in terms of mouse body weights, activities of alanine transaminase (ALT) and aspartate transaminase (AST), and white blood cell counts (FIGS. 14-16). The free PTX-treated mice showed significantly lower white blood cell counts (around 30% of the HBSS group), in agreement with the reported neutropenia or leucopenia. By contrast, the nanocarrier improved PTX's pharmacokinetic profile and biodistribution, resulting in low side toxicity. The hematoxylin and eosin (H&E) staining lacked histological signs of toxicity in major organs with the MMP2-sensitive nanopreparation. However, necrotic areas were clearly present in the MMP2-sensitive nanopreparation-treated tumors (FIG. 17), in agreement with antitumor effects observed in FIGS. 12 and 13. The high therapeutic index of the MMP2-sensitive nanopreparation is most likely a result of the “collaborative” functions including the “stealth” character of PEGylation, the EPR effect, the MMP2-sensitivity, the TATp-mediated intracellular drug delivery, and the enhanced penetration/diffusion.

Most recently, high drug loading micellar nanocarriers have been reported. However, compared to these micelles, which usually load the drug via physical forces between drug and polymer hydrophobic fragments, the risk of drug leakage of the MMP2-sensitive nanopreparation is minimized by the covalent bond between PTX and the polymer (FIG. 22). The most important is that: (1) the PEG2000 and MMP2-sensitive linker in the PTX conjugate allowed the tumor cell-specific cytotoxicity (FIGS. 4-9); the small size and PEG “corona” of the MMP2-sensitive nanopreparation decreased the non-specific tissue distribution/cell internalization (FIGS. 10-13); the combined use of various functions in a “collaborative manner” enhanced nanopreparations' tumor cell-selective internalization, resulting in high anticancer activities and low side effects (FIGS. 10 and 17).

### Example 19

### Biodistribution and Intratumoral Localization

HBSS, the rhodamine-labeled MMP2-sensitive nanopreparation, and its non-sensitive counterpart were intravenously injected in tumor-bearing mice at the dose of 5 mg/Kg PTX. At 2 h post-injection, the tumor and major organs were collected, followed by cell dissociation. The single-cell suspension was analyzed by flow cytometry analysis (FACS). The tumors were sectioned and analyzed by confocal microscopy. To determine the PTX's tissue accumulation, the tissues and blood were homogenized and the PTX was measured by high-performance liquid chromatography (HPLC).

Statistical Analysis.

Data were presented as mean±standard deviation (SD). The difference between the groups was analyzed using a one-way ANOVA analysis by the commercial software PASW® Statistics 18 (SPSS). P<0.05 was considered statistically significant.

While certain embodiments have been illustrated and described, it should be understood that changes and modifications can be made therein in accordance with ordinary skill in the art without departing from the technology in its broader aspects as defined in the following claims.

The embodiments, illustratively described herein, may suitably be practiced in the absence of any element or elements, limitation or limitations, not specifically disclosed herein. Thus, for example, the terms “comprising,” “including,” “containing,” etc., shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to, plus or minus 10% of the particular term.

The use of the terms “a,” “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art, all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

What is claimed is:

1. A co-polymer of formula (I)
A-B-C (I)
or a pharmaceutically acceptable salt thereof, wherein
A comprises a water soluble polymer;
B comprises a matrix metalloprotease (MMP)-cleavable polypeptide;
C is a chemotherapeutic drug or a derivative thereof; and
A is connected to B at a first end through a first covalent bond or a first linking moiety and B is connected to C at a second end through a second covalent bond or a second linking moiety, and wherein the co-polymer is not cross-linked.

2. The co-polymer of claim 1, wherein the co-polymer is of formula (II)
A¹-L¹-B-C (II)
or a pharmaceutically acceptable salt thereof, wherein
A¹ is X—O (R₁O)ₙ—, wherein X is hydrogen, acyl or alkyl, R₁ is C₂-C₈ alkylene, optionally substituted, and n is from 1 to 500;
L¹ is a first linking moiety;
B comprises a MMP-cleavable polypeptide; and
C is a chemotherapeutic drug or a derivative thereof.

3. The co-polymer of claim 2, wherein A¹ is C₁-C₆ alkyl.

4. The co-polymer of claim 2, wherein A¹ is —O(CH₂CH₂O)ₙ— and n is from 1 to 100.

5. The co-polymer of claim 2, wherein L¹ is —CO—, —CH₂CH₂CO— or —SO₂.

6. The co-polymer of claim 1, wherein the MMP-cleavable polypeptide is a MMP2- or MMP9-cleavable polypeptide.

7. The co-polymer of claim 1, wherein the MMP-cleavable polypeptide is —NH-Gly-Pro-Leu-Gly-Ile-Ala-Gly-Gln-CO₂— (SEQ ID NO:1).

8. The co-polymer of claim 1, wherein the chemotherapeutic drug is selected from the group consisting of a hydroxy-containing chemotherapeutic drug, an amino-containing chemotherapeutic drug, and a hydroxy- and amino-containing chemotherapeutic drug.

9. The co-polymer of claim 8, wherein the hydroxy-containing chemotherapeutic drug is selected from the group consisting of Aclacinomycins, Arzoxifene, Batimastat, Broxuridine, Calusterone, Capecitabine, CC-1065, Chromomycins, Diethylstilbestrol, Docetaxel, Doxifluridine, Droloxifene, Dromostanolone, Enocitabine, Epitiostanol, Estramustine, Etanidazole, Etoposide, Fenretinide, Flavopiridol, Formestane, Fosfestrol, Fulvestrant, Gemcitabine, Irinotecan, Melengestrol, Menogaril, Miltefosine, Mitobronitol, Mitolactol, Mopidamol, Nitracrine, Nogalamycin, Nordihydroguaiaretic Acid, Olivomycins, Paclitaxel and other known paclitaxel analogs, Plicamycin, Podophyllotoxin, Retinoic acid, Roquinimex, Rubitecan, Seocalcitol, Temoporfin, Teniposide, Tenuazonic Acid, Topotecan, Valrubicin, Vinblastine, Vincristine and Zosuquidar.

10. (canceled)

11. The co-polymer of claim 8, wherein the amino-containing chemotherapeutic drug is selected from the group consisting of 9-Aminocamptothecin, Aminolevulinic Acid, Amsacrine, Bisantrene, Cactinomycin, Carboquone, Carmofur, Carmustine, Cyclophosphamide, Dacarbazine, Dactinomycin, Demecolcine, Diaziquone, 6-Diazo-5-oxo-L-norleucine (DON), Edatrexate, Efaproxiral, Eflornithine, Eniluracil, Erlotinib, Fluorouracil, Gefitinib, Gemcitabine, Goserelin, Histamine, Ifosfamide, Imatinib, Improsulfan, Lanreotide, Leuprolide, Liarozole, Lobaplatin, Cisplatin, Carboplatin, Lomustine, Lonafarnib, Mannomustine, Melphalan, Methotrexate, Methyl Aminolevulinate, Miboplatin, Mitoguazone, Mitoxantrone, Nilutamide, Nimustine, Nolatrexed, Oxaliplatin, Pemetrexed, Phenamet, Piritrexim, Procarbazine, Raltitrexed, Tariquidar, Temozolomide, Thiamiprine, Thioguanine, Tipifamib, Tirapazamine, 3-Aminopyridine-2-carboxaldehyde thiosemicarbazone, 3-Aminopyridine-4-methyl-2-carboxaldehyde thiosemicarbazone, Trimetrexate, Uracil Mustard, Uredepa and Meturedepa.

12. (canceled)

13. The co-polymer of claim 8, wherein the hydroxy- and amino-containing chemotherapeutic drug is selected from the group consisting of Ancitabine, Anthramycin, Azacitidine, Bleomycins, Bropirimine, Buserelin, Carubicin, Chlorozotocin, Cladribine, Cytarabine, Daunorubicin, Decitabine, Defosfamide, Docetaxel, Doxorubicin, Ecteinascidins, Epirubicin, Gemcitabine, Hydroxyurea, Idarubicin, Marimastat, 6-Mercaptopurine, Pentostatin, Peplomycin, Perfosfamide, Pirarubicin, Prinomastat, Puromycin, Ranimustine, Streptonigrin, Streptozocin, Tiazofurin, Troxacitabine, Vindesine and Zorubicin.

14. The co-polymer of claim 1, wherein the chemotherapeutic drug is Paclitaxel or a Paclitaxel analog.

15. The co-polymer of claim 2, wherein n is from 1 to 50.

16. A composition comprising micelles, wherein the micelles comprise the co-polymer of claim 1.

17. The composition of claim 16, wherein the micelles further comprise an amphiphilic compound.

18. The composition of claim 17, wherein the amphiphilic compound comprises one or more compounds selected from the group consisting of poly(ethylene glycol) (PEG), a phospholipid, and a cell penetrating peptide.

19. (canceled)

20. The composition of claim 18, wherein the phospholipid comprises dioleoylphosphatidyl ethanolamine (DOPE) phosphatidylethanolamine (cephalin) (PE), phosphatidic acid (PA), phosphatidylcholine (PC), 1,2-distearoyl-sn-glycero-3-phosphoethanolamine (DSPE) or phosphatidylserine (PS).

21. (canceled)

22. The composition of claim 18, wherein the cell penetrating peptide is Cys-Tyr-Gly-Arg-Lys-Lys-Arg-Arg-Gln-Arg-Arg-Arg (TATp, SEQ ID NO:15).

23. The composition of claim 17, wherein the amphiphilic compound is one or more of PEG1000-PE and TATp-PEG1000-DOPE.

24. The composition of claim 17, wherein the co-polymer(s) and amphiphilic compound(s) are in a ratio of about 1:0.1 to about 1:10.

25.-26. (canceled)

27. A method for treating cancer in a subject comprising administering to the subject an effective amount of a composition comprising the co-polymer of claim 1.

28. A method for delivering an chemotherapeutic drug into one or more cells of a subject comprising administering to the subject an effective amount of a composition comprising the co-polymer of claim 1.
